SEQUENCE LISTING (7 sequences)

SEQ ID NO: 1 - RNA, 12 nt, Artificial Sequence
  Essential part of crRNA
guuuuagagcua

SEQ ID NO: 2 - RNA, 10 nt, Artificial Sequence
  3′-terminal part of crRNA
ugcuguuuug

SEQ ID NO: 3 - RNA, 60 nt, Artificial Sequence
  Essential part of tracrRNA
uagcaaguuaaaauaaggcuaguccguuaucaacuugaaaaaguggcaccgagucggugc

SEQ ID NO: 4 - protein, 1368 aa, Artificial Sequence [sequence omitted]

SEQ ID NO: 5 - protein, 42 aa, Artificial Sequence [sequence omitted]

SEQ ID NO: 6 - protein, 339 aa, Artificial Sequence
  Human sRAGE protein, wherein the region of first 14 amino acids is N-Terminal His-tag
MRGSHHHHHHGMASAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGEGFDKVREAEDSPQHM

SEQ ID NO: 7 - DNA, 20 bp, Artificial Sequence
  Target site of human AAVS1
gtcaccaatcctgtccctag

(19) United States
(12) Patent Application Publication
(10) Pub. No.: US 2020/0289575 A1
(43) Pub. Date: Sep. 17, 2020

(54) PHARMACEUTICAL COMPOSITION FOR PREVENTING OR TREATING NEUROLOGICAL DISORDERS OR CARDIOVASCULAR DISEASES, COMPRISING SRAGE-SECRETING STEM CELL

(71) Applicant: NSAGE CORP., Incheon (KR)

(72) Inventors: Bonghee Lee, Jeju-si (KR); Delger Bayarsaikhan, Ulaanbaatar, MN (US); Jaeseok Lee, Incheon (KR); Hosseinisalkadeh Seyedghasem, Teheran (IR)

(21) Appl. No.: 16/610,135

(22) PCT Filed: May 2, 2018
(86) PCT No.: PCT/KR2018/005100

§ 371 Date: Feb. 6, 2020

(30) Foreign Application Priority Data
May 2, 2017 (KR) 10-2017-0056433
Jun. 20, 2017 (KR) 10-2017-0078196

(51) Int. Cl. A61K 35/28; A61P 25/28

(52) U.S. Cl. CPC A61K 35/28; A61P 25/28

(57) ABSTRACT

Disclosed are sRAGE-secreting stem cells and a use thereof in preventing and/or treating degenerative disease, such as Parkinson's disease, and/or cardiovascular disease.

### TECHNICAL FIELD

Provided are a sRAGE-secreting stem cell and a use thereof for preventing and/or treating neurologic diseases and/or cardiovascular diseases.

### BACKGROUND ART

Parkinson's disease (PD) is a representative one of the fatal neurodegenerative diseases caused by various factors such as genetic or sporadic causes with toxic drugs and so forth. Patients suffering from PD have movement difficulties due to chronic progressive destruction of the nervous system. Characterized by muscular rigidity, bradykinesia, tremor at rest, and postural instability, the movement difficulties contribute to impaired quality of life. Hence, the effective treatment of PD is very important from the standpoint of providing improved quality of life for patients with PD.

Extensive research has been conducted and is ongoing in order to reveal the cause of PD. According to genetic studies, for example, mutations on specific genes, such as SNCA, PARK2, LRRK2, PINK1, etc., were found in patients with PD. These genes are, in the most part, associated with alpha-synuclein, which is a main component of Lewy bodies. The formation of Lewy bodies in the substantia nigra (SN) makes dopaminergic neuron (DA) cells face apoptosis. In addition, DA cells in SN are found to be damaged under the chronic PD condition, with the activated microglial cells playing an important role in neuronal cell death. If a chronic PD condition is established in the brain, particularly the SN, dopaminergic neurons release cytokines as signaling molecules.

Since the substantia nigra and the corpus striatum (CS) are linked together, DA cells in SN send signals to CS by producing dopamine. Therefore, when the apoptosis of DA cells happens in the area of SN, dopamine is not produced any more from SN, with the consequent discontinuation of supply of CS with signals to react to movement. Continuation of this issue would cause the area to be damaged by disuse atrophy.

Many studies have reported various causes of PD, but failed to propose certain evidences for the damage of the CS area in PD. In spite of intensive research into the neurodegeneration of SN to find the cause of PD, the underlying mechanisms for neuronal cell death in CS still remain uncertain.

As such, the limited research results obtained for causes of PD thus far cannot support a promising therapy for PD.

Albumin, which is a family of proteins most abundantly found in blood plasma, is synthesized primarily in hepatocytes and serves as a main component in most of the extracellular fluids include interstitial fluid, lymph fluid, and cerebrospinal fluid. Since a reduced level of albumin in the body accounts for liver hypofunction and malnutrition, albumins are widely used for clinical treatment of critical conditions in serious patients or vascular collapse in liver cirrhosis patients.

Advanced glycation end products (AGE), which are composites produced mainly by reaction between carbohydrates and free amino acids, are known as chemically very labile and highly reactive, promoting neuronal cell death. In addition, AGE is reported to have increased levels in the brains of elderly persons or aged animals and have an influence on all cells and biological molecules, causing senescence and senescence-related chronic diseases. That is, AGE is associated with the onset of adult diseases including senescence, Alzheimer's disease, renal disease, diabetes mellitus, diabetic vascular complication, diabetic retinopathy, and diabetic neuropathy by increasing vascular permeability, nitrogen oxide-regulated vasodilation impairment, LDL oxidation, release of various cytokines from macrophages or endothelial cells, and oxidative stress.

As described above, AGE is known to increase in tissues of elderly persons or aged animals and to act as a cause of senescence and senescence-related chronic disease. It has thus been proposed in many studies that AGE promotes the death of cells to influence the onset of degenerative diseases or ischemic diseases. In recent, AGE-albumin has been found to predominate in AGES in various diseases, acting as a direct cause of the diseases. There is therefore a desperate need for a technique inhibitory of AGE-albumin.

### DETAILED DESCRIPTION OF THE INVENTION

### Technical Problem

An embodiment provides a sRAGE (soluble Receptor for Advanced Glycation End-products)-secreting stem cell. In one embodiment, the sRAGE-secreting stem cell may be a human sRAGE-secreting stem cell. Another embodiment provides a sRAGE-secreting stem cell having a sRAGE-encoding gene inserted into the genome of a stem cell, for example, a safe harbor site, such as AAVS1, in the genome of a stem cell. The stem cells may be a mesenchymal stem cells, for example, a mesenchymal stem cell derived from umbilical cord blood.

Another embodiment provides a pharmaceutical composition, comprising a sRAGE-secreting stem cell or a sRAGE-secreting stem cell culture, for repressing the secretion of AGE (advanced glycation end-product)-albumin. Another embodiment provides a method for repressing the secretion of AGE-albumin, the method comprising a step of administering a sRAGE-secreting stem cell or a sRAGE-secreting stem cell culture to a subject in need of repressing the secretion of AGE-albumin. The repression against the secretion of AGE-albumin may be repression against the secretion of AGE-albumin in mononuclear phagocytes.

Another embodiment provides a pharmaceutical composition for inhibiting AGE-albumin-induced cell death, which comprises a sRAGE-secreting stem cell or a sRAGE-secreting stem cell culture. Another embodiment provides a method for inhibiting AGE-albumin-induced cell death, the method comprising a step of administering a sRAGE-secreting stem cell or a sRAGE-secreting stem cell culture to a subject in need of inhibiting AGE-albumin-induced cell death. The inhibition of AGE-albumin-induced cell death may be inhibition of AGE-albumin-induced cell death in mononuclear phagocytes.

Another embodiment provides a pharmaceutical composition comprising a sRAGE-secreting stem cell or a sRAGE-secreting stem cell as an effective ingredient for inhibiting apoptosis in a patient suffering from a neurologic disease, for example, a patient with a neurodegenerative disease, such as Parkinson's disease (PD). The composition may inhibit the death of peripheral cells of mononuclear phagocytes, but is not limited thereto. The peripheral cells of mononuclear phagocytes may be neuronal cells and the neuronal cells may be at least one selected from the group consisting of astrocytes, neurons, and dopaminergic neurons, but are not limited thereto.

Another embodiment provides a pharmaceutical composition comprising a sRAGE-secreting stem cell or a sRAGE-secreting stem cell culture as an effective ingredient for prevention and/or treatment of a neurologic disease.

Another embodiment provides a method for repressing the synthesis and/or secretion of AGE (Advanced Glycation End-product)-albumin and/or RAGE (Receptor for Advanced Glycation End-products), inhibiting apoptosis in a patient with a neurologic disease, and/or preventing and/or treating a neurologic disease, the method comprising a step of administering a sRAGE-secreting stem cell or a sRAGE-secreting stem cell culture to a subject in need thereof. The method may further comprise a step of identifying a subject in need of repressing the synthesis and/or secretion of AGE-albumin and/or RAGE, inhibiting apoptosis in a patient with a neurologic disease, and/or preventing and/or treating a neurologic disease, prior to the administering step.

Another embodiment provides a use of a sRAGE-secreting stem cell or a sRAGE-secreting stem cell culture (1) in repressing the synthesis and/or secretion of AGE-albumin and/or RAGE, inhibiting apoptosis in a patient with a neurologic disease, and/or preventing and/or treating a neurologic disease, or (2) in preparing a pharmaceutical composition for repressing the synthesis and/or secretion of AGE-albumin and/or RAGE, inhibiting apoptosis in a patient with a neurologic disease, and/or preventing and/or treating a neurologic disease.

Herein, the term “neurologic disorder/neurologic disease” is intended to encompass all disorders/diseases caused by structural and/or functional injury (impairment), degeneration, and/or pause in the nervous system, that is, the brain, the spinal cord, and/or the nerves. In an embodiment, the neurologic disorder/neurologic disease may be at least one selected from the group consisting of neurodegenerative diseases, such as Parkinson's disease (PD), amyotrophic lateral sclerosis (ALS, Lou Gehrig's disease), frontotemporal dementia (HD), dementia with Lewy bodies (DLB), corticobasal degeneration, multiple system atrophy (MSA), progressive supranuclear palsy (PSP), and Huntington's disease (HD); spinal cord injury; alcoholic poisoning (e.g., alcoholic cerebellar degeneration, alcoholic peripheral neuropathy, and so forth); and stroke.

Another embodiment provides a pharmaceutical composition comprising a sRAGE-secreting stem cell or a sRAGE-secreting stem cell culture as an effective ingredient for prevention or treatment of a cardiovascular disease.

Another embodiment provides a method for prevention or treatment of a cardiovascular disease, the method comprising a step of administering a pharmaceutically effective amount of a sRAGE-secreting stem cell or a sRAGE-secreting stem cell culture to a subject in need thereof.

Another embodiment provides a use of a sRAGE-secreting stem cell or a sRAGE-secreting stem cell culture in preventing or treating a cardiovascular disease or in preparing a pharmaceutical composition for prevention or treatment of a cardiovascular disease.

The cardiovascular disease, which is a disease caused by cardiovascular dysfunction, may be selected from all ischemic cardiovascular diseases, for example, at least one selected from the group consisting of stroke, myocardial infarction, angina pectoris, limb ischemia, hypertension, and arrhythmia, but is not limited thereto.

Another embodiment provides a method for preparation of a sRAGE-secreting stem cell, the method comprising a step of introducing a sRAGE gene into a genome of a stem cell. The step of introducing a sRAGE gene into a genome of a stem cell may be conducted with a complex of an endonuclease (or a nucleic acid molecule coding therefor) and a guide RNA (or a nucleic acid molecule coding therefor). The complex of an endonuclease and a guide RNA may be CRISPR/Cas9 RNP (Ribonucleoprotein; RNA Guided Endonuclease; RGEN).

Another embodiment provides a sRAGE-secreting stem cell prepared by the preparation method.

Another embodiment provides an endonuclease (or nucleic acid molecule coding therefor) and guide RNA (or nucleic acid molecule coding therefor) complex for use in preparing a sRAGE-secreting stem cell, for example, CRISPR/Cas9 RNP.

### Technical Solution

An embodiment provides a sRAGE (soluble Receptor for Advanced Glycation End-products)-secreting stem cell. In one embodiment, the sRAGE-secreting stem cell may be a human sRAGE-secreting stem cell. Another embodiment provides a sRAGE-secreting stem cell having a sRAGE-encoding gene inserted into the genome of a stem cell, for example, a sRAGE-encoding gene inserted into a safe harbor site, such as AAVS1, in the genome of a stem cell. The stem cells may be a mesenchymal stem cells, for example, a mesenchymal stem cell derived from umbilical cord blood.

Another embodiment provides a pharmaceutical composition, comprising a sRAGE-secreting stem cell or a sRAGE-secreting stem cell culture, for repressing the secretion of AGE (advanced glycation end-product)-albumin. Another embodiment provides a method for repressing the secretion of AGE-albumin, the method comprising a step of administering a sRAGE-secreting stem cell or a sRAGE-secreting stem cell culture to a subject in need of repressing the secretion of AGE-albumin. The repression against the secretion of AGE-albumin may be repression against the secretion of AGE-albumin in mononuclear phagocytes.

Another embodiment provides a pharmaceutical composition for inhibiting AGE-albumin-induced cell death (apoptosis), which comprises a sRAGE-secreting stem cell or a sRAGE-secreting stem cell culture. Another embodiment provides a method for inhibiting AGE-albumin-induced cell death, the method comprising a step of administering a sRAGE-secreting stem cell or a sRAGE-secreting stem cell culture to a subject in need of inhibiting AGE-albumin-induced cell death. The inhibition of AGE-albumin-induced cell death may be inhibition of AGE-albumin-induced cell death in mononuclear phagocytes.

Another embodiment provides a pharmaceutical composition comprising a sRAGE-secreting stem cell or a sRAGE-secreting stem cell as an effective ingredient for inhibiting apoptosis in a patient suffering from a neurologic disease. The composition may inhibit the death of peripheral cells of mononuclear phagocytes, but is not limited thereto. The patient suffering from a neurologic disease may be a Parkinson's disease patient. The peripheral cells of mononuclear phagocytes may be neuronal cells and the neuronal cells may be at least one selected from the group consisting of astrocytes, neurons, and dopaminergic neurons, but are not limited thereto.

Another embodiment provides a pharmaceutical composition comprising a sRAGE-secreting stem cell or a sRAGE-secreting stem cell culture as an effective ingredient for prevention and/or treatment of a neurologic disease.

Another embodiment provides a method for repressing the synthesis and/or secretion of AGE (Advanced Glycation End-product)-albumin and/or RAGE (Receptor for Advanced Glycation End-products), inhibiting apoptosis in a patient with a neurologic disease, and/or preventing and/or treating a neurologic disease, the method comprising a step of administering a sRAGE-secreting stem cell or a sRAGE-secreting stem cell culture to a subject in need thereof. The method may further comprise a step of identifying a subject in need of repressing the synthesis and/or secretion of AGE-albumin and/or RAGE, inhibiting apoptosis in a patient with a neurologic disease, and/or preventing and/or treating a neurologic disease, prior to the administering step.

Another embodiment provides a use of a sRAGE-secreting stem cell or a sRAGE-secreting stem cell culture (1) in repressing the synthesis and/or secretion of AGE-albumin and/or RAGE, inhibiting apoptosis in a patient with a neurologic disease, and/or preventing and/or treating a neurologic disease, or (2) in preparing a pharmaceutical composition for repressing the synthesis and/or secretion of AGE-albumin and/or RAGE, inhibiting apoptosis in a patient with a neurologic disease, and/or preventing and/or treating a neurologic disease.

Herein, the neurologic disorder/neurologic disease may encompass all disorders/diseases caused by structural and/or functional injury (impairment), degeneration, and/or pause in the nervous system, that is, the brain, the spinal cord, and/or the nerves. In an embodiment, the neurologic disorder/neurologic disease may be at least one selected from the group consisting of neurodegenerative diseases, such as Parkinson's disease (PD), amyotrophic lateral sclerosis (ALS, Lou Gehrig's disease), frontotemporal dementia (HD), dementia with Lewy bodies (DLB), corticobasal degeneration, multiple system atrophy (MSA), progressive supranuclear palsy (PSP), and Huntington's disease (HD); spinal cord injury; alcoholic poisoning (e.g., alcoholic cerebellar degeneration, alcoholic peripheral neuropathy, and so forth); and stroke.

Another embodiment provides a pharmaceutical composition comprising a sRAGE-secreting stem cell or a sRAGE-secreting stem cell culture as an effective ingredient for prevention or treatment of a cardiovascular disease.

Another embodiment provides a method for prevention or treatment of a cardiovascular disease, the method comprising a step of administering a pharmaceutically effective amount of a sRAGE-secreting stem cell or a sRAGE-secreting stem cell culture to a subject in need thereof.

Another embodiment provides a use of a sRAGE-secreting stem cell or a sRAGE-secreting stem cell culture in preventing or treating a cardiovascular disease or in preparing a pharmaceutical composition for prevention or treatment of a cardiovascular disease.

The cardiovascular disease, which is a disease caused by cardiovascular dysfunction, may be selected from all ischemic cardiovascular diseases, for example, at least one selected from the group consisting of stroke, myocardial infarction, angina pectoris, limb ischemia, hypertension, and arrhythmia, but is not limited thereto.

Another embodiment provides a method for preparation of a sRAGE-secreting stem cell, the method comprising a step of introducing a sRAGE gene into a genome of a stem cell. The step of introducing a sRAGE gene into a genome of a stem cell may be conducted with a complex of an endonuclease (or a nucleic acid molecule coding therefor) and a guide RNA (or a nucleic acid molecule coding therefor). The complex of an endonuclease and a guide RNA may be CRISPR/Cas9 RNP (Ribonucleoprotein; RNA Guided Endonuclease; RGEN).

Another embodiment provides a sRAGE-secreting stem cell prepared by the preparation method.

Another embodiment provides an endonuclease (or nucleic acid molecule coding therefor) and guide RNA (or nucleic acid molecule coding therefor) complex for use in preparing a sRAGE-secreting stem cell, for example, CRISPR/Cas9 RNP.

Another embodiment provides a use of a sRAGE-secreting iPSC in protecting a stem cell administered together therewith (see Example 14 and FIGS. 21a and 21b). The stem cell may be a cell isolated from a biological organism, which is different from and is administered together with the sRAGE-secreting iPSC. In detail, a composition comprising a sRAGE-secreting iPSC for preventing a stem cell is provided. Another embodiment provides a method for protecting a stem cell, the method comprising a step of co-culturing an isolated sRAGE-secreting iPSC and the stem cell isolated. The co-culturing may be conducted in vitro. Another embodiment provides a composition comprising a stem cell therapy product and a sRAGE-secreting iPSC for combination therapy. Another embodiment provides a stem cell therapy method comprising a step of co-administering a stem cell therapy product and a sRAGE-secreting iPSC to a patient in need thereof. The stem cell therapy product and the sRAGE-secreting iPSC may be administered concurrently or regardless of the order thereof. The production of a stem cell may be the protection of the stem cell from AGE-albumin accumulation-induced injury.

Hereinafter, a detailed description will be given of the present disclosure: The patient may be selected from mammals including primates such as humans, apes, and the like and rodents such as rats, mice, and the like, which suffer from neurodegenerative disease and/or cardiovascular disease, cells (brain cells or myocardial or cardiovascular cells) or tissues (brain tissues or cardiac tissues) isolated from the mammals, or cultures thereof. By way of example, selection may be made of a human suffering from neurodegenerative disease and/or cardiovascular disease, brain cells, brain tissues, cardiomyocytes, cardiovascular cells, cardiac tissues isolated therefrom, or a culture of the cells or tissues.

The sRAGE-secreting stem cell provided as an effective ingredient in the disclosure or a pharmaceutical composition comprising the same may be administered via various routes including oral and parenteral routes. For example, the cells or composition may be administered in any convenient way, such as injection, transfusion, implantation, or transplantation into a lesion site (e.g., brain, heart (cardiomyocytes, cardiac vessels, etc.)) of a patient with neurodegenerative disease, or via vessel routes (vein or artery), without any limitation thereto.

The pharmaceutical compositions provided herein may be formulated according to conventional methods into oral dosage forms such as powders, granules, tablets, capsules, suspensions, emulsions, syrups, aerosols, or parenteral dosage forms such as suspensions, emulsions, lyophilized agent, external preparations, suppositories, sterile injectable solutions, implant preparations, and the like.

The amount of the composition of the present disclosure may vary depending on the age, sex, and weight of the subject to be treated, and above all, the condition of the subject to be treated, the specific category or type of cancer to be treated, the route of administration, the nature of the therapeutic agent used, and the sensitivity to specific therapeutic agents, and may be prescribed in consideration thereof. For example, the stem cells may be administered to an Alzheimer's disease patient at a dose of 1×10³-1×10⁹ cells, e.g., 1×10⁴-1×10⁹ cells, 1×10⁴-1×10⁸ cells, 1×10⁵-1×10⁷ cells, or 1×10⁵-1×10⁶ cells per kg of body weight, but is not limited thereto.

The sRAGE may be derived from mammals including primates humans, apes, and the like, and rodents such as rats, mice, and the like. In one embodiment, the sRAGE may be at least one selected from the group consisting of the human sRAGE proteins (GenBank Accession Nos: NP_001127.1 (gene: NM_001136.4) [Q15109-1], NP_001193858.1 (gene: NM_001206929.1) [Q15109-6], NP_001193861.1 (gene: NM_001206932.1) [Q15109-7], NP_001193863.1 (gene: NM_001206934.1) [Q15109-4], NP_001193865.1 (gene: NM_001206936.1) [Q15109-9], NP_001193869.1 (gene: NM_001206940.1) [Q15109-3], NP_001193883.1 (gene: NM_001206954.1) [Q15109-8], NP_001193895.1 (gene: NM_001206966.1) [Q15109-3], and NP_751947.1 (gene: NM_172197.2) [Q15109-2]), but is not limited thereto.

As used herein, the term “stem cell” is intended to encompass all embryonic stem cells, adult stem cells, induced pluripotent stem cells (iPS cells), and progenitor cells. For example, the stem cells may be at least one selected from the group consisting of embryonic stem cells, adult stem cells, induced pluripotent stem cells, and progenitor cells.

Embryonic stem cells are stem cells derived from an embryo and able to differentiate into cells of any tissue.

Induced pluripotent stem cells (iPS cells), also called dedifferentiated stem cells, are embryonic-like pluripotent cells that are generated by injecting a cell differentiation related gene into differentiated somatic cells to reprogram the somatic cells back to a pre-differentiation cell state.

Progenitor cells have a tendency to differentiate into a specific type of cells, but are already more specific than stem cells and are pushed to differentiate into their target cells. Unlike stem cells, progenitor cells undergo limited divisions. The progenitor cells may be derived from mesenchymal stem cells, but are not limited thereto. In the disclosure, progenitor cells fall within the scope of stem cells and unless otherwise stated, “stem cells” are construed to include progenitor cells.

Adult stem cells, which are stem cells derived from the umbilical cord, umbilical cord blood or adult bone marrow, blood, nerves, etc., refer to primitive cells immediately before differentiation into cells of concrete organs. The adult stem cells are at least one selected from the group consisting of hematopoietic stem cells, mesenchymal stem cells, neural stem cells, and the like.

Adult stem cells are difficult to proliferate and are prone to differentiation. Instead, adult stem cells can be used not only to reproduce various organs required by actual medicine, but also to differentiate according to the characteristics of individual organs after transplantation thereto. Hence, adult stem cells can be advantageously applied to the treatment of incurable diseases.

In one embodiment, the adult stem cells may be mesenchymal stem cells (MSC). The term “mesenchymal stem cells”, also called mesenchymal stromal cells (MSC), means multipotent stromal cells that can differentiate into various types of cells, such as osteoblasts, chondrocytes, myocytes, adipocytes, and the like. Mesenchymal stem cells may be selected from pluripotent cells derived from non-marrow tissues such as placenta, umbilical cord, umbilical cord blood, adipose tissues, adult muscles, corneal stroma, and dental pulp from deciduous teeth.

The sRAGE-secreting stem cell may be at least one selected from the group consisting of human-derived sRAGE-secreting mesenchymal stem cells (hereinafter referred to as “human RAGE-secreting MSC”) and human-derived sRAGE-secreting induced pluripotent stem cells (hereinafter referred to as “human sRAGE-secreting iPSC”). In one embodiment, the stem cell may be a human-derived stem cell, for example, a human umbilical cord mesenchymal stem cell or umbilical cord blood mesenchymal stem cell, but are not limited thereto.

The sRAGE-secreting stem cell may be a stem cell, e.g., a mesenchymal stem cell or induced pluripotent stem cell, having a sRAGE-encoding gene inserted to the genome thereof.

In one embodiment, the sRAGE-encoding gene may be inserted into a safe harbor gene site in the genome of the stem cell. A safe harbor gene site is a genomic location where DNA may be damaged (cleaved, and/or deletion, substitution, or insertion of nucleotide(s)) without disrupting cell injury may include, but is not limited to, AAVS1 (adeno-associated virus integration site; e.g., AAVS1 in human chromosome 19 (19q13)).

Insertion (introduction) of the sRAGE-encoding gene into a stem cell genome may be achieved using any genetic manipulation technique that is typically used to introduce a gene into a genome in an animal cell. In one embodiment, the genetic manipulation technique may employ target-specific nuclease. The target-specific nuclease may target such a safe harbor gene site as is described above.

As used herein, the term “target-specific nuclease”, which is also called programmable nuclease, is intended to encompass all types of nucleases (e.g., endonucleases) that recognize and cleave specific sites on target genomic DNA. The target-specific nuclease may be an enzyme isolated from a microbe or a non-naturally occurring enzyme obtained in a recombinant or synthetic manner. The target-specific nuclease may further include an element that is typically used for intracellular delivery in eukaryotic cells (e.g., nuclear localization signal; NLS), but is not limited thereto. The target specific nuclease may be used in the form of a purified protein, a DNA encoding the same, or a recombinant vector carrying the DNA.

For example, the target-specific nuclease may be at least one selected from the group consisting of:

transcription activator-like effector nuclease (TALEN) in which a transcription activator-like (TAL) effector DNA-binding domain, derived from a gene responsible for plant infection, for recognizing a specific target sequence, is fused to a DNA cleavage domain;

zinc-finger nuclease (ZFN);

meganuclease;

RNA-guided engineered nuclease (RGEN), which is derived from the microbial immune system CRISPR, such as Cas proteins (e.g., Cas9, etc.), Cpf1, and the like; and

Ago homolog (DNA-guided endonuclease), but is not limited thereto.

The target-specific nuclease recognizes specific base sequences in the genome of animal and plant cells (i.e., eukaryotic cells), including human cells, to cause double strand breaks (DSBs). The double strand breaks create a blunt end or a cohesive end by cleaving the double strands of DNA. DSBs are efficiently repaired by homologous recombination or non-homologous end-joining (NHEJ) mechanisms within the cell, which allows researchers to introduce desired mutations into on-target sites during this process.

The meganuclease may be included within, but is not limited to, a scope of naturally occurring meganucleases. The naturally occurring meganucleases recognize 15-40 base pair-long sites to be cleaved and are commonly classified into families: LAGLIDADG family, GIY-YIG family, His-Cyst box family, and HNH family. Exemplary meganucleases include I-SceI, I-CeuI, PI-PspI, PI-SceI, I-SceIV, I-CsmI, I-PanI, I-SceII, I-PpoI, I-SceIII, I-CreI, I-TevI, I-TevII, and I-TevIII.

DNA-binding domains from naturally occurring meganucleases, primarily from the LAGLIDADG family, have been used to promote site-specific genome modification in plants, yeasts, Drosophila, mammalian cells, and mice, but this approach has been limited to the modification of either homologous genes that conserve the meganuclease recognition sequence (Monet et al. (1999), Biochem. Biophysics. Res. Common. 255: 88-93) or pre-engineered genomes into which a recognition sequence has been introduced. Accordingly, attempts have been made to engineer meganucleases to exhibit novel binding specificity at medically or biotechnologically relevant sites. In addition, naturally occurring or engineered DNA-binding domains from meganucleases have been operably linked to a cleavage domain from a heterologous nuclease (e.g., Fokl).

The ZFN comprises a zinc finger protein engineered to bind to a target site in a gene of interest and cleavage domain or a cleavage half-domain. The ZFN may be an artificial restriction enzyme comprising a zinc-finger DNA binding domain and a DNA cleavage domain. Here, the zinc-finger DNA binding domain may be engineered to bind to a sequence of interest For example, reference may be made to Beerli et al. (2002) Nature Biotechnol. 20:135-141; Pabo et al. (2001) Ann. Rev. Biochem. 70:313-340; Isalan et al, (2001) Nature Biotechnol. 19: 656-660; Segal et al. (2001) Curr. Opin. Biotechnol. 12:632-637; and Choo et al. (2000) Curr. Opin. Struct Biol. 10:411-416. Compared to a naturally occurring zinc finger protein, an engineered zinc finger binding domain can have a novel binding specificity. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence.

Selection of target sites, and design and construction of fusion proteins (and polynucleotides encoding the same) are known to those skilled in the art and described in detail in U.S. Patent Nos. 2005/0064474 A and 2006/0188987 A, incorporated by reference in their entireties herein. In addition, as disclosed in these and other references, zinc finger domains and/or multi-fingered zinc finger proteins may be linked together using any suitable linker sequences, including, for example, linkers of 5 or more amino acids in length. Reference may be made to U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

Nucleases such as ZFNs also comprise a nuclease active site (cleavage domain, cleavage half-domain) As noted above, the cleavage domain may be heterologous to the DNA-binding domain, for example, such as a zinc finger DNA-binding domain and a cleavage domain from a different nuclease. Heterologous cleavage domains can be obtained from any endonuclease or exonuclease. Exemplary endonucleases from which a cleavage domain can be derived include, but are not limited to, restriction endonucleases and meganucleases.

Similarly, a cleavage half-domain can be derived from any nuclease or portion thereof, as set forth above, which requires dimerization for cleavage activity. In general, two fusion proteins are required for cleavage if the fusion proteins comprise cleavage half-domains Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e g , by dimerizing. Thus, in an embodiment, the near edges of the target sites are separated by 5-8 nucleotides or by 14-18 nucleotides.

However, any integral number of nucleotides or nucleotide pairs can intervene between two target sites (e.g., from 2 to 50 nucleotide pairs or more). Generally, the site of cleavage lies between the target sites.

Restriction endonucleases (restriction enzymes) are present in many species and are capable of binding to DNA (at a recognition site) in a sequence-specific manner and cleaving DNA at or near the site of binding. Certain restriction enzymes (e.g., Type IIS) cleave DNA at sites removed from the recognition site and have separable binding and cleavage domains For example, the Type IIS enzyme Fok I catalyzes double-stranded cleavage of DNA, at 9 nucleotides from its recognition site on one strand and 13 nucleotides from its recognition site on the other. Thus, in one embodiment, fusion proteins comprise the cleavage domain (or cleavage half-domain) from at least one Type IIS restriction enzyme and one or more zinc finger binding domains (which may or may not be engineered).

As used herein, the term “TALEN” refers to a nuclease capable of recognizing and cleaving a target region of DNA. TALEN is a fusion protein comprising a TALE domain and a nucleotide cleavage domain. In the present disclosure, the terms “TAL effector nuclease” and “TALEN” are interchangeably used. TAL effectors are known as proteins that are secreted by Xanthomonas bacteria via their type lit secretion system when they infect a variety of plant species. The protein may be bound to a promoter sequence in a host plant to activate the expression of a plant gene that aids bacterial infection. The protein recognizes plant DNA sequences through a central repetitive domain consisting of various numbers of 34 or fewer amino acid repeats. Accordingly, TALE is considered to be a novel platform for tools in genome engineering. However, in order to construct a functional TALEN with genomic-editing activity, a few key parameters that have remained unknown thus far should be defined as follows: i) the minimum DNA-binding domain of TALE, the length of the spacer between the two half-sites constituting one target region, and the linker or fusion junction that links the Fold nuclease domain to dTALE.

The TALE domain of the present disclosure refers to a protein domain that binds nucleotides in a sequence-specific manner via one or more TALE-repeat modules. The TALE domain includes, but is not limited to, at least one TALE-repeat module, and more specifically, 1 to 30 TALE-repeat modules. In the present disclosure, the terms “TAL effector domain” and “TALE domain” are interchangeable. The TALE domain may include half of the TALE-repeat module. As concerns the TALEN, reference may be made to Patent Publication No. WO/2012/093833 or U.S. Patent No. 2013-0217131 A of which the entire contents are incorporated by reference in their entireties herein.

In one embodiment, insertion (or introduction) of the sRAGE-encoding gene into a stem cell genome may be achieved using a target-specific nuclease (RGEN derived from CRISPR). The target-specific nuclease may comprise:

(1) an RNA-guided nuclease (or a DNA coding therefore or a recombinant vector carrying the coding DNA), and

(2) a guide RNA capable of hybridizing with (or having a complementary nucleotide sequence to) a target site (e.g., a region of 15 to 30, 17 to 23, or 18 to 22 consecutive nucleotides in a safe harbor gene such as AAVS1) in a target gene (e.g., a safe harbor site such as AAVS1), or a DNA coding therefor (or a recombinant vector carrying the coding DNA).

For example, the target-specific nuclease may be at least one selected from all nucleases that can recognize specific sequences of target genes and have nucleotide cleavage activity to incur indel (insertion and/or deletion) in the target genes.

In one embodiment, the target-specific nuclease may be at least one selected from the group consisting of nucleases (e.g., endonucleases) included in the type II and/or type V CRISPR system, such as Cas proteins (e.g., Cas9 protein (CRISPR (Clustered regularly interspaced short palindromic repeats) associated protein 9)), Cpf1 protein (CRISPR from Prevotella and Francisella 1), etc. In this regard, the target-specific nuclease further comprises a target DNA-specific guide RNA for guiding to a target site on a genomic DNA. The guide RNA may be an RNA transcribed in vitro, for example, RNA transcribed from double-stranded oligonucleotides or a plasmid template, but is not limited thereto. The target-specific nuclease may act in a ribonucleoprotein (RNP) form in which the nuclease is associated with guide RNA to form a ribonucleic acid-protein complex (RNA-Guided Engineered Nuclease), in vitro or after transfer to a body (cell).

The Cas protein, which is a main protein component in the CRISPR/Cas system, accounts for activated endonuclease or nickase activity.

The Cas protein or gene information may be obtained from a well-known database such as GenBank at the NCBI (National Center for Biotechnology Information). By way of example, the Cas protein may be at least one selected from the group consisting of:

a Cas protein derived from Streptococcus sp., e.g., Streptococcus pyogenes, for example, Cas9 protein (i.e., SwissProt Accession number Q99ZW2 (NP_269215.1));

a Cas protein derived from Campylobacter sp., e.g., Campylobacter jejuni, for example, Cas9 protein;

a Cas protein derived from Streptococcus sp., e.g., Streptococcus thermophiles or Streptococcus aureus, for example, Cas9 protein;

a Cas protein derived from Neisseria meningitidis, for example, Cas9 protein;

a Cas protein derived from Pasteurella sp., e.g., Pasteurella multocida, for example, Cas9 protein; and

a Cas protein derived from Francisella sp., e.g., Francisella novicida, for example, Cas9 protein, but is not limited thereto.

According to one embodiment, in a case where the Cas9 protein is derived from Streptococcus pyogenes, the PAM sequence may be 5′-NGG-3′ (N is A, T, G, or C) and the nucleotide sequence site to be cleaved (target site) may be a consecutive 17 bp- to 23 bp-long, for example, 20 bp-long nucleotide sequence located adjacent to the 5′- and/or 3′-end of the 5′-NGG-3′ sequence in a target gene.

According to another embodiment, in a case where the Cas9 protein is derived from Campylobacter jejuni, the PAM sequence may be 5′-NNNNRYAC-3′ (N's are each independently A, T, C or G, R is A or G, and Y is C or T) and the nucleotide sequence site to be cleaved (target site) may be a consecutive 17 bp- to 23 bp-long, for example, 22 bp- to 23 bp-long nucleotide sequence located adjacent to the 5′- and/or 3′-end of the NNNNRYAC-3′ sequence in a target gene.

According to another embodiment, in a case where the Cas9 protein is derived from Streptococcus thermophiles, the PAM sequence may be 5′-NNAGAAW-3′ (N's are each independently A, T, C, or G, and W is A or T) and the nucleotide sequence site to be cleaved (target site) may be a consecutive 17 bp- to 23 bp-long, for example, 21 bp- to 23 bp-long nucleotide sequence located adjacent to the 5′- and/or 3′-end of the NNAGAAW-3′ sequence in a target gene.

According to another embodiment, in a case where the Cas9 protein is derived from Neisseria meningitidis, the PAM sequence may be 5′-NNNNGATT-3′(N's are each independently A, T, C or G) and the nucleotide sequence site to be cleaved (target site) may be a consecutive 17 bp- to 23 bp-long, for example, 21 bp- to 23 bp-long nucleotide sequence located adjacent to the 5′- and/or 3′-end of the 5′-NNNNGATT-3′ sequence in a target gene.

According to another embodiment, in a case where the Cas9 protein is derived from Streptocuccus aureus, the PAM sequence may be 5′-NNGRR(T)-3′ (N's are each independently A, T, C or G, R is A or G, and (T) means an optional sequence included therein) and the nucleotide sequence site to be cleaved (target site) may be a consecutive 17 bp- to 23 bp-long, for example, 21 bp- to 23 bp-long nucleotide sequence located adjacent to the 5′- and/or 3′-end of the 5′-NNGRR(T)-3′ sequence in a target gene.

The Cpf1 protein, which is an endonuclease in a new CRISPR system distinguished from the CRISPR/Cas system, is small in size relative to Cas9, requires no tracrRNA, and can act with the guidance of single guide RNA. In addition, the Cpf1 protein recognizes a thymine-rich PAM (protospacer-adjacent motif) sequence and cleaves DNA double strands to form a cohesive end (cohesive double-strand break).

By way of example, the Cpf1 protein may be derived from Candidatus spp., Lachnospira spp., Butyrivibrio spp., Peregrinibacteria, Acidominococcus spp., Porphyromonas spp., Prevotella spp., Francisella spp., Candidatus Methanoplasma, or Eubacterium spp., e.g., from Parcubacteria bacterium (GWC2011_GWC2_44_17), Lachnospiraceae bacterium (MC2017), Butyrivibrio proteoclasiicus, Peregrinibacteria bacterium (GW2011_GWA_33_10), Acidaminococcus sp. (BV3L6), Porphyromonas macacae, Lachnospiraceae bacterium (ND2006), Porphyromonas crevioricanis, Prevotella disiens, Moraxella bovoculi (237), Smiihella sp. (SC_KO8D17), Leptospira inadai, Lachnospiraceae bacterium (MA2020), Francisella novicida (U112), Candidatus Methanoplasma termitum, Candidatus Paceibacter, Eubacterium eligens, etc., but is not limited thereto.

In a case where Cpf1 protein is used as the endonuclease, the PAM sequence is 5′-TTN-3′ (N is A, T, C, or G) and the nucleotide sequence site to be cleaved (target site) may be a consecutive 17 bp- to 23bp-long, for example, 21 bp- to 23 bp-long nucleotide sequence located adjacent to the 5′- and/or 3′-end of the 5′-TTN-3′ sequence in a target gene.

The target-specific nuclease may be isolated from microbes or may be an artificial or non-naturally occurring enzyme as obtained by recombination or synthesis. For use, the target-specific nuclease may be in the form of an mRNA pre-described or a protein pre-produced in vitro or may be included in a recombinant vector so as to be expressed in target cells or in vivo. In an embodiment, the target-specific nuclease (e.g., Cas9, Cpf1, etc.) may be a recombinant protein made with a recombinant DNA (rDNA). The term “recombinant DNA” means a DNA molecule formed by artificial methods of genetic recombination, such as molecular cloning, to bring together homologous or heterologous genetic materials from multiple sources. For use in producing a target-specific nuclease by expression in a suitable organism (in vivo or in vitro), recombinant DNA may have a nucleotide sequence that is reconstituted with optimal codons for expression in the organism which are selected from codons coding for a protein to be produced.

The target-specific nuclease used herein may be a mutant target-specific nuclease in an altered form. The mutant target-specific nuclease may refer to a target-specific nuclease mutated to lack the endonuclease activity of cleaving double strand DNA and may be, for example, at least one selected from among mutant target-specific nucleases mutated to lack endonuclease activity but to retain nickase activity and mutant target-specific nucleases mutated to lack both endonuclease and nickase activities. As such, the mutation of the target-specific nuclease (e g., amino acid substitution, etc.) may occur at least in the catalytically active domain of the nuclease (for example, RuvC catalyst domain for Cas9). In an embodiment, when the target-specific nuclease is a Streptococcus pyogenes-derived Cas9 protein (SwissProt Accession number Q99ZW2(NP_269215.1); SEQ ID NO: 4), the mutation may be amino acid substitution at one or more positions selected from the group consisting of a catalytic aspartate residue (e.g., aspartic acid at position 10 (D10) for SEQ ID NO: 4, etc.), glutamic acid at position 762 (E762), histidine at position 840 (H840), asparagine at position 854 (N854), asparagine at position 863 (N863), and aspartic acid at position 986 (D986) on the sequence of SEQ ID NO: 4. A different amino acid to be substituted for the amino acid residues may be alanine, but is not limited thereto.

In another embodiment, the mutant target-specific nuclease may be a mutant that recognizes a PAM sequence different from that recognized by wild-type Cas9 protein. For example, the mutant target-specific nuclease may be a mutant in which at least one, for example, all of the three amino acid residues of aspartic acid at position 1135 (D1135), arginine at position 1335 (R1335), and threonine at position 1337 (T1337) of the Streptococcus pyogenes-derived Cas9 protein are substituted with different amino acids to recognize NGA (N is any residue selected from among A, T, G, and C) different from the PAM sequence (NGG) of wild-type Cas9.

In one embodiment, the mutant target-specific nuclease may have the amino acid sequence (SEQ ID NO: 4) of Streptococcus pyogenes-derived Cas9 protein on which amino acid substitution has been made for:

(1) D10, H840, or D10+H840;

(2) D1135, R1335, T1337, or D1135+R1335+T1337; or

(3) both of (1) and (2) residues.

As used herein, the term “a different amino acid” means an amino acid selected from among alanine, isoleucine, leucine, methionine, phenylalanine, proline, tryptophan, valine, asparagine, cysteine, glutamine, glycine, serine, threonine, tyrosine, aspartic acid, glutamic acid, arginine, histidine, lysine, and all variants thereof, exclusive of the amino acid retained at the original mutation positions in wild-type proteins. In one embodiment, “a different amino acid” may be alanine, valine, glutamine, or arginine.

As used herein, the term “guide RNA” refers to an RNA that includes a targeting sequence hybridizable with a specific base sequence (target sequence) of a target site in a target gene and functions to associate with a nuclease, such as Cas proteins, Cpf1, etc., and to guide the nuclease to a target gene (or target site) in vitro or in vivo (or in cells).

The guide RNA may be suitably selected depending on kinds of the nuclease to be complexed therewith and/or origin microorganisms thereof.

For example, the guide RNA may be at least one selected from the group consisting of:

CRISPR RNA (crRNA) including a region (targeting sequence) hybridizable with a target sequence;

trans-activating crRNA (tracrRNA) including a region interacting with a nuclease such as Cas protein, Cpf1, etc.; and

single guide RNA (sgRNA) in which main regions of crRNA and tracrRNA (e.g., a crRNA region including a targeting sequence and a tracrRNA region interacting with nuclease) are fused to each other.

In detail, the guide RNA may be a dual RNA including CRISPR RNA (crRNA) and trans-activating crRNA (tracrRNA) or a single guide RNA (sgRNA) including main regions of crRNA and tracrRNA.

The sgRNA may include a region (named “spacer region”, “target DNA recognition sequence”, “base pairing region”, etc.) having a complementary sequence (targeting sequence) to a target sequence in a target gene (target site), and a hairpin structure for binding to a Cas protein. In greater detail, the sgRNA may include a region having a complementary sequence (targeting sequence) to a target sequence in a target gene, a hairpin structure for binding to a Cas protein, and a terminator sequence. These moieties may exist sequentially in the direction from 5′ to 3′, but without limitations thereto. So long as it includes main regions of crRNA and tracrRNA and a complementary sequence to a target DNA, any guide RNA can be used in the present disclosure.

For editing a target gene, for example, the Cas9 protein requires two guide RNAs, that is, a CRISPR RNA (crRNA) having a nucleotide sequence hybridizable with a target site in the target gene and a trans-activating crRNA (tracrRNA) interacting with the Cas9 protein. In this context, the crRNA and the tracrRNA may be coupled to each other to form a crRNA:tracrRNA duplex or connected to each other via a linker so that the RNAs can be used in the form of a single guide RNA (sgRNA). In one embodiment, when a Streptococcus pyogenes-derived Cas9 protein is used, the sgRNA may form a hairpin structure (stem-loop structure) in which the entirety or a part of the crRNA having a hybridizable nucleotide sequence is connected to the entirety or a part of the tracrRNA including an interacting region with the Cas9 protein via a linker (responsible for the loop structure).

The guide RNA, specially, crRNA or sgRNA, includes a targeting sequence complementary to a target sequence in a target gene and may contain one or more, for example, 1-10, 1-5, or 1-3 additional nucleotides at an upstream region of crRNA or sgRNA, particularly at the 5′ end of sgRNA or the 5′ end of crRNA of dual RNA. The additional nucleotide(s) may be guanine(s) (G), but are not limited thereto.

In another embodiment, when the nuclease is Cpf1, the guide RNA may include crRNA and may be appropriately selected, depending on kinds of the Cpf1 protein to be complexed therewith and/or origin microorganisms thereof.

Concrete sequences of the guide RNA may be appropriately selected depending on kinds of the nuclease (Cas9 or Cpf1) (i.e., origin microorganisms thereof) and are an optional matter which could easily be understood by a person skilled in the art.

In an embodiment, when a Streptococcus pyogenes-derived Cas9 protein is used as a target-specific nuclease, crRNA may be represented by the following General Formula 1:

(General Formula 1)
5′-(N_{cas9})ₗ-(GUUUUAGAGCUA)-(X_{cas9})m-3′

wherein:

N_{cas9} is a targeting sequence, that is, a region determined according to a sequence at a target site in a target gene (i.e., a sequence hybridizable with a sequence of a target site), 1 represents a number of nucleotides included in the targeting sequence and may be an integer of 15 to 30, 17 to 23 or 18 to 22, for example, 20;

the region including 12 consecutive nucleotides (GUUUUAGAGCUA; SEQ ID NO: 1) adjacent to the 3′-end of the targeting sequence is essential for crRNA;

X_{cas9} is a region including m nucleotides present at the 3′-terminal site of crRNA (that is, present adjacent to the 3′-end of the essential region); and

m may be an integer of 8 to 12, for example, 11 wherein the m nucleotides may be the same or different and are independently selected from the group consisting of A, U, C, and G.

In an embodiment, the X_{cas9} may include, but is not limited to, UGCUGUUUUG (SEQ ID NO: 2).

In addition, the tracrRNA may be represented by the following General Formula 2:

(General Formula 2)
5′-(Y_{cas9})ₚ-(UAGCAAGUUAAAAUAAGGCUAGUCCGUUAUCAACU
UGAAAAAGUGGCACCGAGUCGGUGC)-3′

wherein,

the region represented by 60 nucleotides (UAGCAAGUUAAAAUAAGGCUAGUCCGUUAUCAACUUGAAAAAGUGGCACC GAGUCGGUGC) (SEQ ID NO: 3) is essential for tracrRNA,

Y_{cas9} is a region including p nucleotides present adjacent to the 3′-end of the essential region, and

p is an integer of 6 to 20, for example, 8 to 19 wherein the p nucleotides may be the same or different and are independently selected from the group consisting of A, U, C, and G.

Further, sgRNA may form a hairpin structure (stem-loop structure) in which a crRNA moiety including the targeting sequence and the essential region of the crRNA and a tracrRNA moiety including the essential region (60 nucleotides) of the tracrRNA are connected to each other via an oligonucleotide linker (responsible for the loop structure). In greater detail, the sgRNA may have a hairpin structure in which a crRNA moiety including the targeting sequence and an essential region of crRNA is coupled with the tracrRNA moiety including the essential region of tracrRNA to form a double-strand RNA molecule with connection between the 3′ end of the crRNA moiety and the 5′ end of the tracrRNA moiety via an oligonucleotide linker.

In one embodiment, the sgRNA may be represented by the following General Formula 3:

(General Formula 3)
5′-(N_{cas9})ₗ-(GUUUUAGAGCUA)-(oligonuc1eotide
linker)-(UAGCAAGUUAAAAUAAGGCUAGUCCGUUAUCAACUUGAA
AAAGUGGCACCGAGUCGGUGC)-3′

wherein (N_{cas9})₁ is a targeting sequence defined as in General Formula 1.

The oligonucleotide linker included in the sgRNA may be 3-5 nucleotides long, for example 4 nucleotides long in which the nucleotides may be the same or different and are independently selected from the group consisting of A, U, C, and G.

The crRNA or sgRNA may further contain 1 to 3 guanines (G) at the 5′ end thereof (that is, the 5′ end of the targeting sequence of crRNA).

The tracrRNA or sgRNA may further comprise a terminator inclusive of 5 to 7 uracil (U) residues at the 3′ end of the essential region (60 nt long) of tracrRNA.

The target sequence for the guide RNA may be about 17 to about 23 or about 18 to about 22, for example, 20 consecutive nucleotides adjacent to the 5′ end of PAM (Protospacer Adjacent Motif (for S. pyogenes Cas9, 5′-NGG-3′ (N is A, T, G, or C)) on a target DNA.

As used herein, the term “the targeting sequence” of guide RNA hybridizable with the target sequence for the guide RNA refers to a nucleotide sequence having a sequence complementarity of 50% or higher, 60% or higher, 70% or higher, 80% or higher, 90% or higher, 95% or higher, 99% or higher, or 100% to a nucleotide sequence of a complementary strand to a DNA strand on which the target sequence exists (i.e., a DNA strand having a PAM sequence (5′-NGG-3′ (N is A, T, G, or C))) and thus can complimentarily couple with a nucleotide sequence of the complementary strand.

In another embodiment, when the target-specific nuclease is a Cpf1 system, the guide RNA (crRNA) may be represented by the following General Formula 4:

(General Formula 4)
5′-n1-n2-A-U-n3-U-C-U-A-C-U-n4-n5-n6-n7-G-U-
A-G-A-U-(Ncpf1)q-3′

wherein,

n1 is null or represents U, A, or G,

n2 represents A or G,

n3 represents U, A, or C,

n4 is null or represents G, C, or A,

n5 represents A, U, C, or G, or is null,

n6 represents U, G, or C,

n7 represents U or G,

Ncpf1 is a targeting sequence including a nucleotide sequence hybridizable with a target site on a target gene and is determined depending on the target sequence of the target gene, and

q represents a number of nucleotides included therein and may be an integer of 15 to 30.

The target sequence (hybridizing with crRNA) of the target gene is a 15 to 30 (e.g., consecutive) nucleotide-long sequence adjacent to the 3′ end of PAM (5′-TTN-3′ or 5′-TTTN-3′; N is any nucleotide selected from A, T, G, and C.

In General Formula 4, the 5 nucleotides from the 6^{th} to the 10^{th} position from the 5′ end (5′ terminal stem region) and the 5 nucleotides from the 15^{th} (16^{th} when n4 is not null) to the 19^{th} (20^{th} when n4 is not null) from the 5′ end are complementary to each other in the antiparallel manner to form a duplex (stem structure), with the concomitant formation of a loop structure composed of 3 to 5 nucleotides between the 5′ terminal stem region and the 3′ terminal stem region.

For the Cpf1 protein, the crRNA (e.g., represented by General Formula 4) may further comprise 1 to 3 guanine residues (G) at the 5′ end.

In crRNA sequences for Cpf1 proteins available from microbes of Cpf1 origin, 5′ terminal sequences (exclusive of targeting sequence regions) are illustratively listed in Table 1.

TABLE 1

| | 5 Terminal Sequence |
|---|---|
| | (5′-3′) of guide |
| Microbe of Cpf1 origin | RNA (crRNA) |
| Parcubacteria bacterium GWC2011_GWC2_44_17 (PbCpf1) | AAAUUUCUACU-UUUGUAGAU |
| Peregrinibacteria bacterium GW2011_GWA_33_10 (PeCpf1) | GGAUUUCUACU-UUUGUAGAU |
| Acidaminococcus sp. BVBLG (AsCpf1) | UAAUUUCUACU-CUUGUAGAU |
| Porphyromonas macacae (PmCpf1) | UAAUUUCUACU-AUUGUAGAU |
| Lachnospiraceae bacterium ND2006 (LbCpi1) | GAAUUUCUACU-AUUGUAGAU |
| Porphyromonas crevioricanis(PcCpf1) | UAAUUUCUACU-AUUGUAGAU |
| Prevotella disiens (PdCpf1) | UAAUUUCUACU-UCGGUAGAU |
| Moraxella bovoculi 237 (MbCpf1) | AAAUUUCUACUGUUUGUAGAU |
| Leptospira inadai (LiCpf1) | GAAUUUCUACU-UUUGUAGAU |
| Lachnospiraceae bacterium MA2020 (Lb2Cpf1) | GAAUUUCUACU-AUUGUAGAU |
| Francisella novicida U112 (FnCpf1) | UAAUUUCUACU-GUUGUAGAU |
| Candidatus Methanoplasma termitum (CMtCpf1) | GAAUCUCUACUCUUUGUAGAU |
| Eubacterium eligens (EeCpf1) | UAAUUUCUACU--UUGUAGAU |

(-: denotes the absence of any nucleotide)

As used herein, the term “nucleotide sequence” hybridizable with a gene target site refers to a nucleotide sequence having a sequence complementarity of 50% or higher, 60% or higher, 70% or higher, 80% or higher, 90% or higher, 95% or higher, 99% or higher, or 100% to a nucleotide sequence (target sequence) of the gene target site (hereinafter used in the same meaning unless otherwise stated. The sequence homology can be using a typical sequence comparison mean (e.g., BLAST)).

In the method, the transduction of the guide RNA and the RNA-guide endonuclease (e.g., Cas9 protein) into cells may be performed by directly introducing the guide RNA and the RNA-guide endonuclease into cells with the aid of a conventional technique (e.g., electroporation, etc.) or by introducing one vector (e.g., plasmid, viral vector, etc.) carrying both a guide RNA-encoding DNA molecule and a RNA-guide endonuclease-encoding gene (or a gene having a sequence homology of 80% or greater, 85% or greater, 90% or greater, 96% or greater, 97% or greater, 98% or greater, or 99% or greater thereto) or respective vectors carrying the DNA molecule or the gene into cells or through mRNA delivery.

In one embodiment, the vector may be a viral vector. The viral vector may be selected from the group consisting of negative-sense single-stranded viruses (e.g., influenza virus) such as retrovirus, adenovirus, parvovirus (e.g., adeno-associated virus (AAV)), corona virus, and orthomyxovirus; positive-sense single-stranded RNA viruses such as rhabdovirus (e.g., rabies virus and vesicular stomatitis virus), paramyxovirus (e.g., measles virus and sendai virus), alphavirus, and picornavirus; and double-stranded DNA viruses such as herpes virus (e.g., herpes simplex virus type 1 and 2, Epstein-Barr virus, cytomegalovirus), and adenovirus; poxvirus (e.g., vaccinia); fowlpox; and canarypox.

A vector carrying the Cas9 protein, the guide RNA, a ribonucleoprotein containing both of them, or at least one thereof may be delivered into a body or cells, using a suitable one of well-known techniques such as electroporation, lipofection, viral vector, nanoparticles, and PTD (protein translocation domain) fusion protein. The Cas9 protein and/or guide RNA may further include a typically useful nuclear localization signal (NLS) for the intranuclear translocation of the Cas9 protein, the guide RNA, or the ribonucleoprotein containing both of them.

As used herein, the term “cleavage” in a target site means the breakage of the covalent backbone in a polynucleotide. The cleavage includes enzymatic or chemical hydrolysis of a phosphodiester bond, but is not limited thereto, and may be performed by various other methods. Cleavage may be possible on both single strands and double strands. The cleavage of a double-strand may result from the cleavage of the two distinct single strands, with the consequent production of blunt ends or staggered ends.

Parkinson's disease (PD) is a progressive degenerative disorder of the central nervous system. Until recently, the underlying mechanisms for neuronal cell death were poorly understood. In the description, the mechanisms of neuronal cell death in PD are unveiled and sRAGE-secreting stem cells (for example, human Umbilical Cord Blood derived Mesenchymal Stem cells (hUCB-MSCs)) are found to make neuronal cell death and behavioral disorder rehabilitation in PD animal models. In one embodiment provided herein, sRAGE-secreting hUCB-MSCs are transplanted into the Corpus striatum of rotenone-induced PD animal models which are then subjected to behavioral test, morphological analysis, and immunohistochemical experiments to determine neuronal cell death and movement recovery. Leading to the present disclosure, the results thus obtained suggest that sRAGE-secreting stem cells have advantageous effects on neurodegenerative disease, including symptom alleviation (reduction), progression inhibition, and/or therapeutic outcomes. sRAGE-secreting stem cells can bring about a better therapeutic outcome in neurodegenerative disease thanks to the continual sRAGE secretion in synergy with the inhibitory effect of the stem cells (e.g., UCB-MSC) themselves on neuronal cell death (neuroprotective effect) in a brain area (e.g., striatum region).

As the extracellular domain of RAGE, sRAGE is in a soluble form. Because the active site of sRAGE is the same as in RAGE, sRAGE can bind with specific ligands such as AGE, S100, and so forth and compete with RAGE for binding with a ligand.

AGE-RAGE Relevance

It has been reported in many literatures that when the ligands of RAGE bind on the RAGE of target cells, they become apoptotic and lead to death eventually. When AGE-albumin binds thereto, RAGE is activated, upregulating the expression of genes associated with apoptosis. This event occurs in other organs in addition to the brain. AGE-RAGE binding acts as a critical cause of cell death in cells of various types. Thus, blockage of AGE-RAGE binding can protect cells from apoptosis.

Generation of sRAGE-Secreting UCB-MSC

sRAGE-secreting stem cells (e.g., UCB-MSC, iPSC, etc.) have many advantages. When being continuously secreted from cells, sRAGE proteins last for a longer period of time than the normal recombinant proteins around the injection site. In addition, the employment of stem cells as the cells secreting sRAGE protein can bring about more advantages because the secreted sRAGE acts in synergy with the stem cells around the injected site. Therefore, a stem cell is one of the most suitable candidates applicable to sRAGE-secreting cell.

In one embodiment, the sRAGE-secreting stem cell for use in PD treatment may be a sRAGE-secreting UCB-MSC or iPSC. In this regard, the sRAGE-secreting stem cell may be a UCB-MSC or iPSC in the first passage after transfection with a sRAGE-encoding gene, which secretes the highest level of sRAGE, but is not limited thereto.

Relationship of AGE-RAGE with Alzheimer's Disease (AD), Alcoholism, and PD

PD animal models have a high accumulation of AGE-albumin, which results in neuronal cell death by AGE-RAGE binding in the CS area. In the present disclosure, the animal models treated with sRAGE or sRAGE-secreting UCB-MSC (or sRAGE-secreting iPSC) is found to rehabilitate the neuronal cell death as analyzed by behavior tests (rotarod and pole tests). Particularly, the sRAGE or sRAGE-secreting UCB-MSC-administered group shows a high blocking effect on AGE-RAGE binding. Hence, sRAGE or sRAGE-secreting UCB-MSC was found to have a better therapeutic potential to protect neuronal cells against apoptosis. Inter alia, the population of neuronal cells in the CS and SN regions of PD animal models was larger when sRAGE or sRAGE-secreting UCB-MSC was administered thereto than in control PD animal models (without treatment with sRAGE or sRAGE-secreting UCB-MSC), indicating that sRAGE-secreting UCB-MSC has a strong neuroprotective performance.

Basis Mechanism for Protective Effect on Neuronal Cell Death

Mitogen-Activated Protein Kinase

Mitogen-activated protein kinase (MAPK) is a protein kinase found in eukaryotes only. Mitogen-activated protein kinases are catalytically inactive in their base form. In order to become active, they require phosphorylation events in their activation loops. The underlying signaling pathway of PD was examined by observing the following classical MAP kinases: ERK1/2, JNK, p38, and phosphorylated forms thereof. As a result of the observation, p38, Erk1/2, and JNK proteins were revealed to be contributors to the cell death mechanism and are thus inferred to be involved in the PD progression pathway.

Bax

To test the effect of sRAGE on the AGE-RAGE dependent pathway, observation was made of Bax. The expression of Bax was increased after AGE-albumin treatment. However, upon co-treatment with sRAGE and AGE-albumin, the expression level of Bax was slightly decreased, indicating that sRAGE protects the cells from apoptosis by blocking AGE-RAGE binding.

Meanwhile, limitations are imparted to the therapeutic application of sRAGE proteins to PD due to the in-vivo half-life thereof. To solve this problem, the present disclosure employs a sRAGE-secreting stem cell (i.e., UCB-MSC or iPSC) to continuously secrete sRAGE proteins.

The secretion level of sRAGE from transfected UCB-MSC was observed to be the highest in the first passage and to decline in the next generations. Cellular transplantation in PD animal models was performed by stereotaxic surgery. Behavior performance of the PD animal models was evaluated in rotarod and pole tests. In the behavior performance tests, the sRAGE-secreting stem cell-treated group was found to significantly improved in movement ability, compared untreated PD groups. In addition, histological analysis revealed that sRAGE-secreting stem cells have protective effect against cell death in the corpus striatum.

In order to examine a mechanism associated with such protective activity, observation was made of protein expression levels in main signaling pathways of PD. Particularly, attention was paid to the expression levels of MAPK proteins and phosphorylated forms thereof. As a result, the neuronal cell death is associated with p38, Erk1/2, and JNK in the MAPK pathway.

In addition, the inhibition of cardiomyocyte or myocyte cell death induction according to the present disclosure is characterized by suppressing the synthesis or secretion of AGE-albumin in mononuclear phagocytes to inhibit the induction of cell death in cells around the mononuclear phagocytes.

Cell death is largely divided into necrosis and apoptosis. Necrosis is the death of cells caused by stimuli such as bums, bruises, poisons and the like, which is known as accidental death of cells. In the case of necrosis, water is introduced from the outside of the cell, causing the cell to expand and then be destroyed. Conventionally, all cell deaths were considered necrosis. However, over the past 30 years, cells have been known to have triggers for spontaneous death. This active cell death, controlled by genes, is apoptosis. Necrosis occurs disorderly over long periods of time, whereas apoptosis occurs in a short time and orderly. Apoptosis begins as cells shrink. Thereafter, gaps occur between adjacent cells, and within the cells, DNA is regularly cut and fragmented. Finally, the whole cell is also fragmented into apoptotic bodies and then eaten by nearby cells, leading to death. Apoptosis is responsible for shaping the body during development, and in adults it is responsible for renewing normal cells or removing abnormal cells. Cell death caused by genetic programs in the process of development and differentiation in an animal's body is called programmed cell death (PCD). Programmed cell death is when a lethal gene begins to move at some stage of development and the cell dies. In the case of humans, the hands or feet are shaped like a spatula in the early stages of the fetus, and there is no gap between the toes or fingers. Degenerative diseases are known to accompany these two types of cells.

In the present disclosure, cell death is preferably limited to cells around mononuclear phagocytes. The cells around mononuclear phagocytes include cardiomyocytes, but are not limited thereto.

The suppression of synthesis or secretion of AGE-albumin may be achieved using at least one selected from the group consisting of albumin siRNA, an albumin antibody, an AGE antibody, an AGE-albumin antibody, and an AGE-albumin synthesis inhibitor.

In the present disclosure, sRAGE-secreting stem cells that can continuously secrete sRAGE (soluble receptor for AGE), which is a kind of antibodies, to inhibit the toxic function of AGE-albumin are generated and used for preventing cardiomyocyte or myocyte death and treating cardiovascular diseases such as myocardial infarction.

### Advantageous Effect

AGE-RAGE dependent cell death contributes to neurodegeneration of CS and SN in PD when the chronic condition was continued. sRAGE prevents neuronal cell death by blocking AGE-RAGE binding. Therefore, a sRAGE secreting stem cell may be one of very effective therapeutic approaches to cure neurodegenerative diseases such as PD and so forth.

In addition, AGE-albumin is synthesized and secreted by macrophages in myocardial infarction or lower limb ischemia models and the synthesis and secretion of AGE-albumin is caused by oxidative stress and induces cell death. Therefore, the sRAGE-secreting stem cell of the present disclosure can be advantageously used for preventing and treating cardiovascular disease such as myocardial infarction, lower limb ischemia, and so forth.

### BRIEF DESCRIPTION OF THE DRAWINGS

FIG. 1 is an illustrative schematic view showing a cleavage map of pZDonor-AAVS1 puromycin vector (A) and an insertion state of a sRAGE coding sequence (B).

FIG. 2 is a schematic view explaining a gene insertion mechanism using target gene transfection and CRISPR/Cas9 RNP.

FIG. 3 shows the secretion of sRAGE proteins from UCB-MSC as analyzed by western blotting against a Flag antibody in the conditioned media of the sRAGE (labeled with Flag)-transfected UCB-MSC cell line (A) and in the densitometry quantitated from the intensity of A by Image J software (B).

FIG. 4 shows maintaining times (seconds) of the control (normal, untreated), the PD group (untreated PD animal model), the sRAGE-treated group (sRAGE-treated PD animal model), and the sRAGE UCB-MSC-treated group (sRAGE-secreting UCB-MSC-treated PD animal model) in the rotarod test for examining animal behaviors (student T-test (p<0.05)).

FIG. 5 shows maintaining times (seconds) of the control (normal, untreated), the PD group (untreated PD animal model), the sRAGE-treated group (sRAGE-treated PD animal model), and the sRAGE UCB-MSC-treated group (sRAGE-secreting UCB-MSC-treated PD animal model) in the pole test for examining animal behaviors (student T-test (p<0.05)).

FIG. 6 shows population changes of neuronal cells in the SN regions of the control (normal, untreated), the PD group (untreated PD animal model), and the sRAGE UCB-MSC-treated group (sRAGE-secreting UCB-MSC-treated PD animal model) as represented by cresyl violet staining images (A) (Bar=100 um) and in a graph of cell counts quantitated by image J software (B).

FIG. 7 shows population changes of neuronal cells in the CS region of the control (normal, untreated), the PD group (untreated PD animal model), and the sRAGE UCB-MSC-treated group (sRAGE-secreting UCB-MSC-treated PD animal model) as represented by cresyl violet staining images (A) (Bar=100 um) and in a graph of cell counts quantitated by image J software (B).

FIG. 8 shows distribution of co-localized AGE and activated microglial cells in the striatum of the control (normal, untreated), the PD (untreated PD animal model), and the sRAGE UCB-MSC (sRAGE-secreting UCB-MSC untreated PD animal model) as represented by fluorescence images of double immunostaining against AGE (green) and Iba-1 (red, activated microglial cell marker), wherein the merged images indicate the co-localization of AGE, ALB, and Iba1 mostly in striatum area of PD brain (rotenone treated mouse brain) (Scale bar: white=50 um, yellow=20 um).

FIG. 9 shows the inhibition of sRAGE against AGE-albumin binding to RAGE as analyzed for cell viability of HT22 cells (neuronal cell lines) in the AGE-albumin-treated group (AA), the AGE-albumin/sRAGE co-treated group (AA-sRAGE), and the untreated group (control) by MIT assay (cell viability expressed as percentages relative to the measurement for the control; OD measured at 570 nm).

FIG. 10 shows levels of MAPK proteins in the CS areas of the control (normal, untreated), the PD group (untreated PD animal model), the sRAGE-treated group (sRAGE-treated PD animal model), and the sRAGE UCB-MSC-treated group (sRAGE-secreting UCB-MSC-treated PD animal model), as measured by western blotting analysis (standard protein: beta-actin).

FIG. 11 shows the concurrence of the increase in the number of macrophagocytes and dead cardiomyocytes in the myocardial infarction rat models, FIG. 11a shows increased populations of macrophages in fluorescence images (upper) and a densitometry graph (lower), and FIG. 11b shows cardiomyocyte death in fluorescence images (upper) and a densitometry graph (lower).

FIG. 12 shows changes in the synthesis and secretion of AGE-albumin around macrophages in the heart tissues of the myocardial infarction rat model, as analyzed by immunohistochemical staining against the antibodies.

FIG. 13 shows the increase of the synthesis and secretion of AGE-albumin in human macrophages upon hypoxic stimulation, as analyzed by ELISA.

FIG. 14a provides fluorescence images showing the increase in the expression of RAGE in primary human cardiomyocytes after administration of AGE-albumin thereto and the decrease in the expression of RAGE after co-administration of sRAGE and FIG. 14b shows the involvement of pSAPK/JNK and p38 in the MAPK pathway responsible for the RAGE signaling, as analyzed by immunoblot assay.

FIG. 15a is a schematic diagram of a vector structure for use in generating sRAGE-secreting mesenchymal stem cells, FIG. 15b show the secretion of sRAGE from the sRAGE-secreting mesenchymal stem cells as analyzed by western blotting and ELISA, and FIG. 15c shows a fluorescent staining result of the secretion in fluorescence images.

FIG. 16 shows the increase of indel frequency by CRISPR/Cas9 RNP in Jurkat cells, wherein the CRISPR/Cas9 RNP is prepared to deliver a vector for generating sRAGE-secreting cells.

FIG. 17 shows degrees of fibrosis in the heart tissues of the myocardial infarction rat models before and after treatment with sRAGE-MSC as analyzed by staining.

FIGS. 18a and 18b show that cell death increased with the increase of RAGE expression in myocytes of the lower limb ischemia model, and decreased after sRAGE administration.

FIGS. 19a to 19c shows characteristics of sRAGE-secreting iPSCs,

FIG. 19a is a schematic diagram of an expression vector for use in generating sRAGE-secreting iPSCs,

FIG. 19b is an image of electrophoresis accounting for a PCR product of sRAGE coding gene transfected into iPSCs with the aid of pZDonor-AAVS1 vector, and

FIG. 9b depicts the expression and secretion levels of sRAGE, as measured by western blotting and ELISA.

FIGS. 20a to 20c shows an protective effect of the sRAGE-secreting iPSC (sRAGE-iPSC) on acute myocardial infarction in images visualizing Masson′ trichrome staining result (a), as calculated for % of fibrosis area and infarcted wall thickness in LV area (b) (*, p<0.05, **, p<0.01, ***, p<0.001), and in fluorescence images of RAGE expression in the heart tissues treated with GFP, VEGF, ANG1 or sRAGE-iPSC as analyzed by immunohistochemistry (c).

FIGS. 21a and 21b shows an protective effect of the sRAGE-secreting iPSC on stem cells in terms of TUNEL (Terminal deoxynucleotidyl transferase dUTP nick end labeling) change after co-treatment with AGE-albumin (AA) and sRAGE-iPSC (21a) and RAGE expression level in iPSCs co-cultured with sRAGE-secreting iPSC after treatment with PBS, AA, and AGE-albumin, as measured by western blotting assay (21b).

### MODE FOR CARRYING OUT THE INVENTION

Hereinafter, the present disclosure will be described in more detail with reference to Examples, which are merely illustrative and are not intended to limit the scope of the present disclosure. It is apparent to those skilled in the art that the Examples described below may be modified without departing from the essential gist of the disclosure.

[Effect on Neurodegenerative Disease (Parkinson's Disease)]

### REFERENCE EXAMPLES

1. Generation of PD Mouse Model

Animal experiments were performed using C57BL/6N mice (20-22 gm). Eight-week-old male mice were randomly divided and housed in cages at a density of five per case and allowed to freely access food and water, with a 12/12 hrs light/dark cycle under a temperature-controlled environment. All the animal protocols described in the disclosure were approved by the Center of Animal Care and Use (CACU) ethical board. To establish a suitable PD model, a suspension of rotenone (Sigma-Aldrich) in 0.5% (w/v) carboxymethyl cellulose (CMC) was orally administered once a day at a dose of 30 mg/kg for two months. Weights of the mice were monitored every week.

2. Stem Cell Culture

Selection was made of umbilical cord blood-derived mesenchymal stem cells (UCB-MSCs, Medi-post) as stem cells to be treated for a PD animal model. UCB-MSCs were grown in a-MEM medium (DMEM, Gibco® Life Technologies Corp.) supplemented with 10% (w/v) fetal bovine serum (FBS, Gibco® Life Technologies Corp.) and 1% (w/v) penicillin and streptomycin (Sigma-Aldrich). These cells were maintained at 37° C. in a humidified air under 5% CO₂. For culturing UCB-MSCs, 100-mm² dishes were used, and the cells were subcultured at 80% confluence. Incubation with Trypsin ETDA (Typsin ETDA, Gibco® Life Technologies Corp) at 37° C. for 5 min detached the cells from the dishes.

3. Generation of Soluble RAGE (sRAGE)-Secreting UCB-MSC

To generate sRAGE-secreting UCB-MSC, transfection was performed with mRNA Zinc Finger Nuclease (Sigma-Aldrich), which is designed to target a safe harbor site of AAVS1. The transfection of UCB-MSC was done by nucleofection in the following conditions: two consecutive shock of 1000V, 30 ms pulse width. After transfection, the cells were seeded at a density of 8×10⁵ cells/well into 6-well plates. The transfected cells were stabilized by incubation at 37° C. for seven days, with the medium freshly changed daily.

4. Stereotaxic Surgery and Tissue Preparation

After 30 days of oral administration of rotenone, the animals were randomly divided into five groups: control (normal mice, untreated); PD mice, α-MEM administered; PD mice, sRAGE administered; PD mice, UCB-MSC administered; and PD mice, sRAGE-secreting UCB-MSC administered. The animals were anesthetized by intraperitoneal injection of a 3:1 mixture of Zoletil 50 (Virbac) and Rompun (Bayer Korea) at a dose of 1 ml/kg prior to surgical procedures. A mouse was mounted on a stereotaxic apparatus (Stoelting Co). According to the Atlas of Paxinos and Watson (Atlas), drugs were unilaterally injected into the right CS anterior and posterior 0.4, medial and lateral 1.8, dorsal and ventral from Bregma 3.5 mm). The injection was performed with the aid of an automatic microinjector (KD Scientic) equipped with a 26-guage Hamilton syringe. Using the automatic microinjector, 3 μl of 10 μM sRAGE was slowly injected at a speed of 1 μl per minute. Then, the syringe was slowly withdrawn and the surgical region was closed before topical treatment with an antibiotic. In 3 μl of alpha-MEM medium free of FBS and antibiotics, 1×10⁶ cells were prepared. To examine the drug injection effect on neuronal cells, perfusion was performed under anesthesia through the heart with 50 ml of 1× PBS, followed by 50 ml of a cold fixative containing 4% (w/v) paraformaldehyde (PFA). After perfusion, the brain was remove and fixed for 5 hours in 4% PFA before storage overnight in a 20% (w/v) sucrose solution. The cryoprotected brain blocks were sectioned into 10-μm thick slices on a cryostat.

5. Immunostaining

Frozen sections of the mouse brain was washed five times with 1× PBS and incubated with a protein-specific antibody. Non-specific binding of antibodies was blocked by normal goat, rabbit, or horse serum (Vector laboratories). Following overnight incubation at 4° C. with the primary antibody, the samples were washed with 1× PBS and then incubated with a secondary antibody at room temperature for one hour. For counterstaining of the nuclei, the samples were stained with DAPI (4′6-diamino-2-phenilindole, 1 μg/ml, Sigma-Aldrich) for 20 seconds. After washing with 1× PBS, coverslips were mounted on a glass slide using Vectashield mounting medium (Vector Laboratories). Analysis was made on an LSM710 confocal microscope (Carl Zeiss).

Primary antibodies used for immunostaining are listed in Table 2, below:

TABLE 2

| Antigen | Host | Company | Application |
|---|---|---|---|
| AGE | Rabbit | abcam (ab23722) | 1 to 1000 |
| lba1 | Goat | abcam (ab5076) | 1 to 1000 |

Secondary antibodies used for immunostaining are listed in Table 3, below:

TABLE 3

| Antigen | Host | Company | Application |
|---|---|---|---|
| Alexa fluor | Donkey | life science | 1 to 500 |
| 555 donkey | | (A21432) | |
| anti-goat IgG | | | |
| Alexa fluor | Donkey | lifes cience | 1 to 500 |
| 488 donkey | | (A21206) | |
| anti-rabbit IgG | | | |

6. Cresyl Violet Staining

Frozen sections of the mouse brain were dried at room temperature for 5 minutes, washed five times with 1× PBS for 10 minutes, and then incubated in graded ethanol (95% ethanol 15 minutes, 70% ethanol 1 minute, 50% ethanol 1 minute). After washing with distilled water, the brain tissues were stained with 0.5% cresyl violet acetate (Sigma-Aldrich) solution for 12 minutes, treated with distilled water (1 minute), 50% ethanol (1 minute), 70% ethanol (2 minutes), 95% ethanol (2 times 2 minutes), 100% ethanol (1 minute), and finally cleaned with xylene (5 minutes). The stained slides were mounted with DPX mounting medium (Sigma-Aldrich) for histological analysis.

7. Western Blotting

Brain tissues were prepared with RIPA lysis buffer (AMRESCO) and added with 1× protease inhibitor (ROCHE), followed by sonication. The tissues were centrifuged at 14000×g for 20 minutes at 4° C. Total protein concentrations were measured by BCA (Life Technologies) according to the manufacturer's protocol. Equal amounts (20 μg) of proteins were separated on a 10% polyacrylamide gel (Life Technologies) and transferred to a PVDF membrane (Millipore Corp.). Proteins were detected with protein-specific antibodies. ECL (Animal Genetics Corp.) detection reagent was used to visualize the immunoreactive proteins on the membrane.

Primary antibodies use for western blotting are listed in Table 4, below:

TABLE 4

| Antigen | Host | Company | Application |
|---|---|---|---|
| Flag | Rabbit | sigma aldrich (F742S) | 1 to 1000 |
| Bax | Rabbit | cell signalling (2774S) | 1 to 1000 |
| RAGE | | | |
| SAPK/JNK | Rabbit | cell signalling (9252S) | 1 to 500 |
| pSAPK/JNK | Rabbit | cell signalling (9251S) | 1 to 500 |
| ERK1/2 | Rabbit | cell signalling (9102S) | 1 to 500 |
| pERK1/2 | Rabbit | cell signalling (4377S) | 1 to 500 |
| p38 | Rabbit | cell signalling (9212S) | 1 to 500 |
| pp38 | Rabbit | cell signalling (9211S) | |
| β-actin | | | |

Secondary antibodies used for western blotting are listed in Table 5, below:

TABLE 5

| Antigen | Host | Company | Application |
|---|---|---|---|
| Peroxidase labeled | Goat | Vector | 1 to 10000 |
| anti-rabbit IgG | | (PI-1000) | |
| (H + L) | | | |
| Peroxidase labeled | Horse | Vector | 1 to 10000 |
| anti-mouse IgG | | (PI-2000) | |
| (H + L) | | | |

8. MTT Assay

HT22 cells (ATCC) were seeded at a density of 2×10³/well into 96-well plates. Thereafter, the cells were treated for 12 hours with AGE-albumin (Sigma-Aldrich) (50 nM). For one hour before 12 hours of treatment with AGE-albumin, the cells were incubated with sRAGE (cat. RD172116100, Biovendor; SEQ ID NO: 6) (50 nM). Cell death was evaluated by MTT assay (3-2,5-diphenyltetrazolium, Sigma-Aldrich). Living cells reduce the yellow MIT compound into purple formazan, which is soluble in dimethyl sulfoxide (Me₂SO). In each well, the cells were incubated for 2 hours with the MTT compound at 0.5 mg/ml and then added with DMSO (Sigma-Aldrich). The intensity of blue staining in the culture medium was measured at 540 nm and 570 nm using a spectrophotometer and was expressed as proportional amounts of living cells.

9. Behavior Test

9.1. Rotarod Test

A rotarod test was performed using an accelerating rotarod (UGO Basile Accelerating Rotarod). In this regard, mice were placed on rotating drums (3 cm in diameter) and measured for the time that each animal was able to maintain its balance on the rod. The speed of the rotarod was 15-16 rpm.

9.2. Pole Test

A pole test was performed with reference to Fleming et al (Neuroscience. 2006 Nov. 3; 142(4): 1245-1253). The stick (1 cm in diameter, 35 cm in height) was vertically attached onto the floor. Mice were placed on the top of the stick standing on the floor and measured for the time when they reached the bottom. After the mice were let to make two training trials, the time measurement was made for the third trial.

Statistical Analysis

All experimental data were presented as the mean±standard deviation (SD). Statistical significance was evaluated using Student's t-test and p<0.05 was considered as significant.

### Example 1

### Characterization of sRAGE-Secreting UCB-MSCs

1-1. Construction of Donor sRAGE Vector

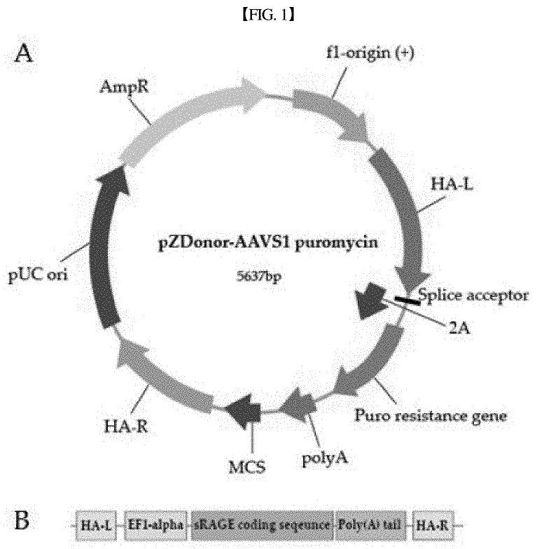

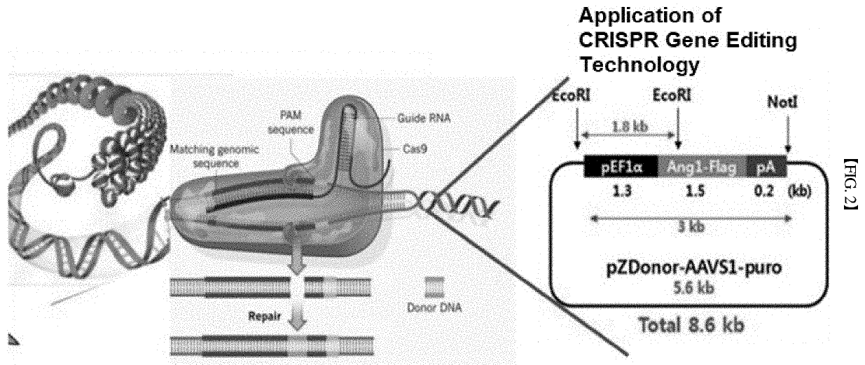

A sRAGE (cat. RD172116100, Biovendor; SEQ ID NO: 6) coding sequence (GenBank Accession No. NM_001206940.1) was prepared and incorporated into AAVS1 pZDonor vector (Sigma Aldrich; FIG. 1A). The vector is 5637 bp long, with HA-L and HA-R for homologous recombination established therein. Having sequences exactly identical to the AAVS1 site, the genes promote the natural repair system (homologous recombination). A homologous sequence insert may be incorporated into the chromosome of UCB-MSC in order to knock a specific gene sequence (sRAGE coding sequence) therein. The Multiple Cloning Sites (MCS) account for various restriction enzyme sites at which the sRAGE coding sequence is inserted into the AAVS1-pZDonor.

1-2. Preparation of Plasmid for Generation of sRAGE-Secreting UCB-MSCs

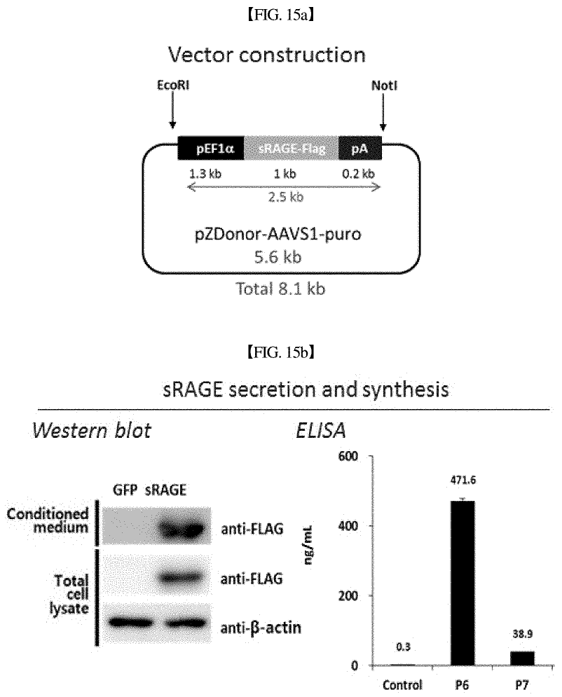

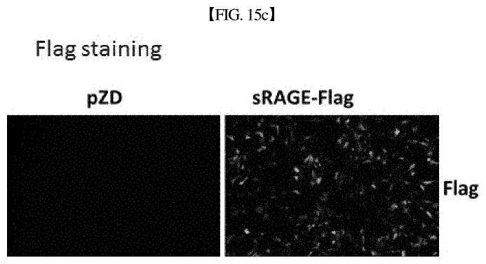

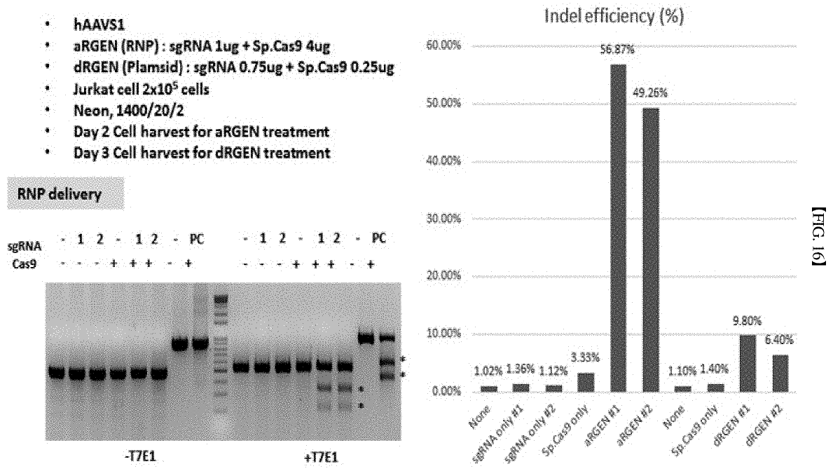

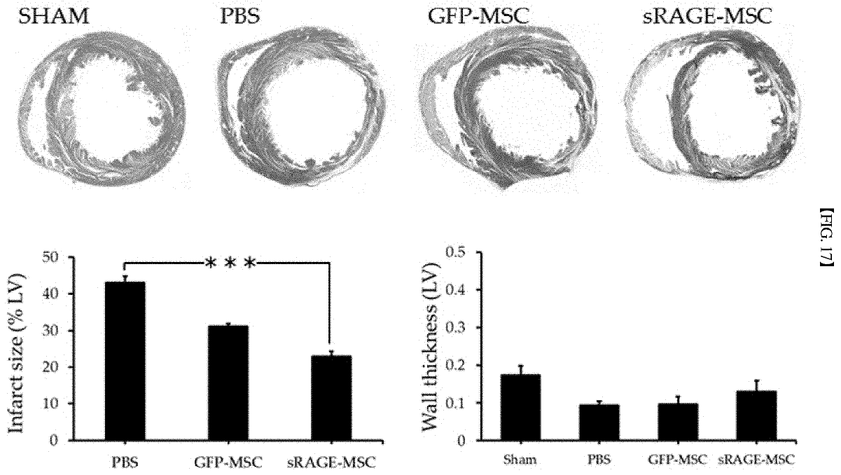

For use in generating sRAGE-secreting UCB-MSCs, an insert was composed of an EF1-alpha promoter, sRAGE coding sequence (SEQ ID NO: 6; flagged in order to facilitate the analysis of sRAGE), and a polyA signal (see FIGS. 1B and 15a). The human EF1-alpha promoter and the polyA signal were amplified from EF1-alpha-AcGFP-C1 (Clontech) and pcDNA3.1 vector (Invitrogen), respectively. EcoRI and NotI restriction sites were used to incorporate the insert into the AAVS1-pZDonor plasmid.

FIG. 1 shows the p/Donor-AAVS1 puromycin vector, along with the insertion information of the sRAGE coding sequence.

1-3. Introduction of sRAGE Coding Sequence into Target Gene in UCB-MSCs by Using CRISPR/Cas9 RNP

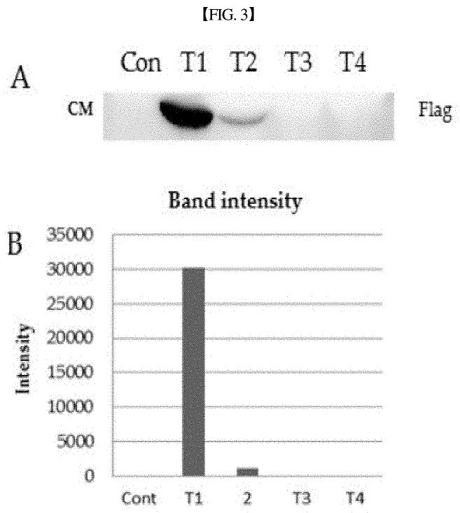

Using an electroporator, AAS1 gene-targeting mRNA CRISPR/Cas9 RNP (ToolGen, Inc; Cas9: Streptococcus pyogenes derived (SEQ ID NO: 4), and AAVS1 target site of sgRNA: 5′-gtcaccaatcctgtccctag-3′ (SEQ ID NO: 7)) was transfected into human UCB-MSCs (CEFObio, Seoul, Korea). After being introduced into cells, the mRNA CRISPR/Cas9 RNP is translated into CRISPR/Cas9 RNP protein. CRISPR/Cas9 RNP gene editing technology is schematically depicted in FIG. 2. The sgRNA has the following nucleotide sequence:

5′-(target sequence)-(GUUUUAGAGCUA; SEQ ID NO:
1)-(nucleotide linker)-(UAGCAAGUUAAAAUAAGGCUAGUC
CGUUAUCAACUUGAAAAAGUGGCACCGAGUCGGUGC; SEQ ID NO:
3)-3′

(the target sequence is the sequence modified from the AAVS1 target site sequence of SEQ ID NO: 7 by converting ‘T’ to “U” and the nucleotide linker has the sequence of GAAA).

In this regard, 10 μl of the sRAGE sequence (used in the form of the vector prepared in Example 1-2) and transfect substrates were used for nucleofection in the following condition; 1050 volts, pulse width 30, pulse number 2, NEON Microporator (Thermo Fisher Scientific, Waltham, Mass.). After being seeded onto a 60 mm-culture dish (BD Biosciences, San Jose, Calif.), 10⁶ cells were stabilized at 37° C. in a 5% CO₂ incubator for 7 days before injection. The medium was freshly changed daily.

The UCB-MSCs prepared to have the sRAGE coding gene introduced into the AAS1 gene thereof were passaged to afford cells of passage numbers 1-4 (T1, T2, T3, and T4): Passage 1 after Transfection (T1), Passage 2 after Transfection (T2), Passage 3 after Transfection (T3), and Passage 4 after Transfection (T4).

1-4. Characterization of sRAGE-Secreting Human UCB-MSC

Because sRAGE protein is secreted outside the sRAGE-secreting UCB-MSCs, the secretion level of sRAGE was measured by western blotting for the conditioned medium where the cells had been cultured (Reference Example 7). The sRAGE protein secreted from the cells was detected with a Flag antibody.

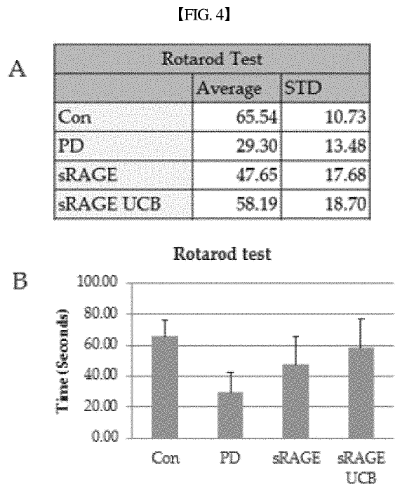

For the control (UCB-MSC, sRAGE coding gene not introduced), T1, T2, T3, and T4, the western blotting results and the densitometry analysis results of band intensities quantitated using Image J software are depicted in FIG. 3. The band intensity was measured to be 0, 30174.41, 1061.7, 0, and 0 for control, T1, T2, T3, and T4, respectively. The intensity of T1 band was 28.4-fold higher than that of T2 band.

### Example 2

### Neuroprotection and Movement Improvement Effect of sRAGE-Secreting UCB-MSC

2-1. Rotarod Test

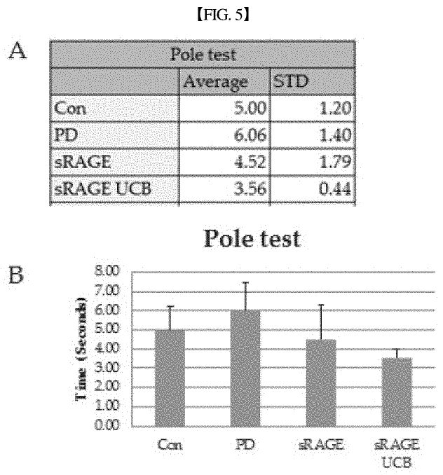

To examine changes of the PD mice in the movement ability, a Rotarod test was performed (Reference Example 9.1). The results are depicted in FIG. 4. The average maintaining period of time was recorded as 65.54±10.73 seconds for control (normal mice), 29.30±13.48 seconds for PD mice (untreated), 47.65±17.68 seconds for sRAGE-treated PD mice, and 58.19±18.70 seconds for sRAGE-secreting UCB-MSC treated PD mice. As shown in FIG. 4, the movement ability was remarkably increased in both sRAGE-secreting UCB-MSC treated mice and sRAGE treated mice. Particularly, treatment with sRAGE-secreting UCB-MSC recovered the movement ability of PD mice to a similar level to normal mice.

2-2. Pole Test

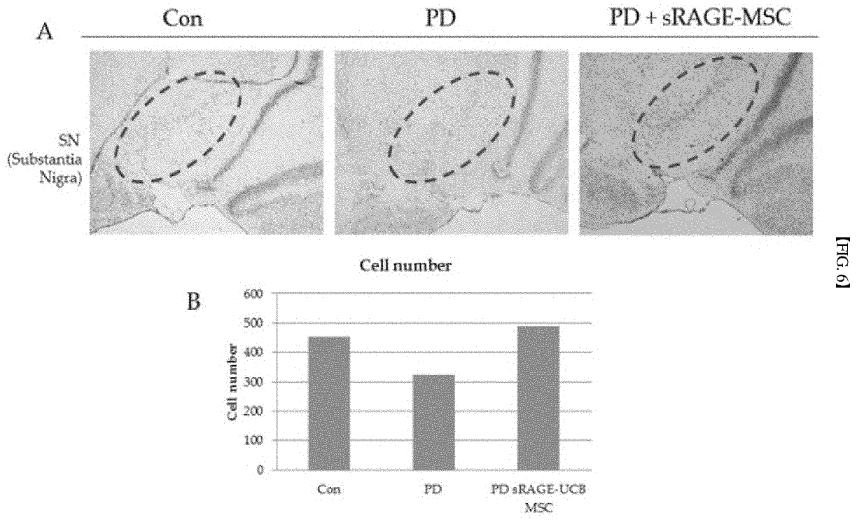

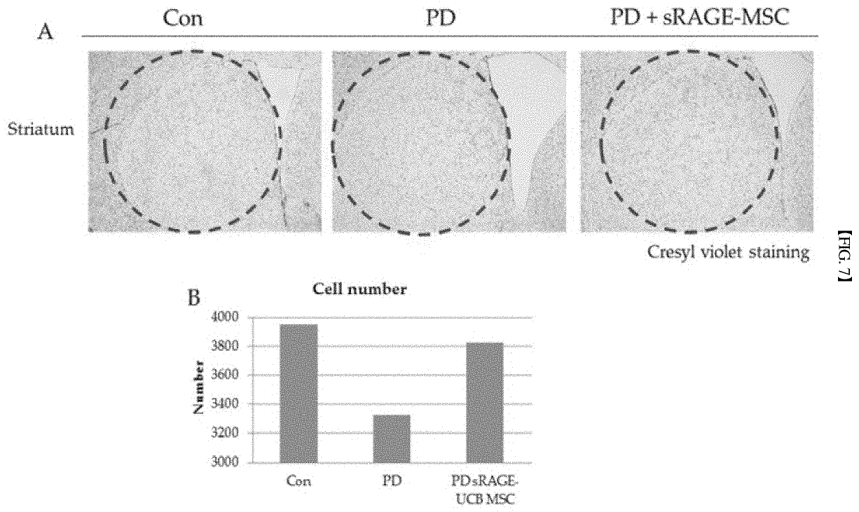

Recovery of animal behavior was also measured by a pole test (Reference Example 9.2). The test results are shown in FIG. 5. The average maintaining periods of time were measured to be 5.00±1.20 seconds for control (normal mice), 6.06±1.40 seconds for PD mice (untreated), 4.52±1.79 seconds for sRAGE-treated PD mice, and 3.56±0.44 seconds for sRAGE-secreting UCB-MSC PD mice (ten mice in each group). As shown in FIG. 5, the behavior recovery was remarkably improved in sRAGE-secreting UCB-MSC- and sRAGE-treated mice, compared to the control group.

2-3. Histological Analysis of Mouse Brain

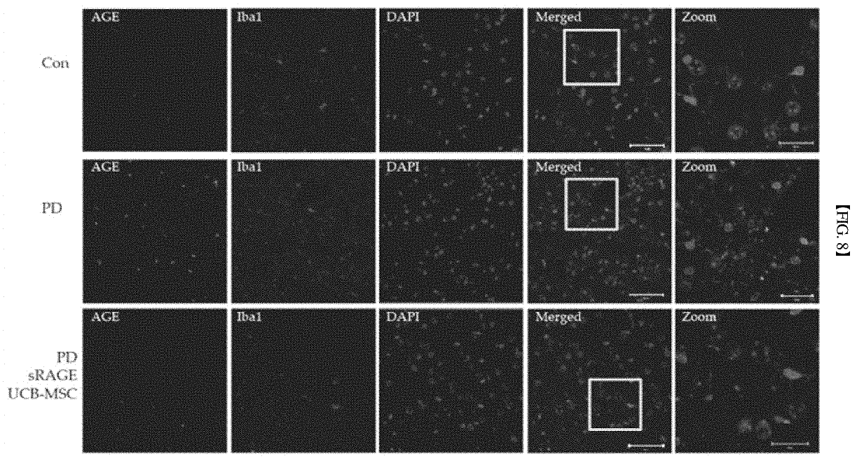

To examine cell death in various brain regions, cresyl violet staining (Reference Example 6) was performed for the SN and CS regions of mice of the following three groups and the images obtained from the staining were analyzed using Image J software to count neuronal cells. The results are given in FIGS. 6 (SN region) and 7 (CS region): control (normal mice), PD mice (untreated), and sRAGE secreting UCB-MSC treated PD mice.

As shown in FIG. 6A (SN region), neuronal cells were stained violet and single dots represent respective single neuronal cells. Most of dopaminergic neurons appeared to exist in the SN region. The cells in the control group were counted up to 453. In contrast, the cell population in the untreated PD mice was reduced to 127 cells. After sRAGE secreting UCB-MSC treatment, the cell population in PD mice was drastically increased to 489 cells. This data implies that sRAGE-secreting UCB-MSC has a remarkable neuroprotective effect in the SN region.

As shown in FIG. 7A (CS region), neuronal cells were stained violet and single dots represent respective single neuronal cells. Cells in the control group were counted up to 3949 while the cell population in the untreated PD mice was reduced to 3329. sRAGE-secreting UCB-MSC treatment drastically increased the cell population to 3822 cells in the PD mice. This data implies that sRAGE-secreting UCB-MSC has a remarkable neuroprotective effect in the CS region.

2-4. Localization of Activated Microglial Cell in CS of PD Mouse Brain

To investigate the relation between AGE formation and microglial activation, immunohistochemical staining was performed on the brain CS (Corpus striatum) region in the mice of the following three groups (Reference Example 5): control (normal mice), PD mice (untreated), and sRAGE-secreting UCB-MSC-treated PD mice. The results are depicted in FIG. 8. As shown in FIG. 8, AGE (green) was almost not observed in the mouse brain of the control group while AGE signals were detected mainly in the CS region of the PD brain and Iba1 (red, activated microglial cell marker) was also localized in the PD brain. Higher signals were detected across the striatum of PD mice than the control brain. These results indicate that more AGE and more activated microglial cells are generated in PD conditions. As can be understood in the merged image of FIG. 8, Iba1 was co-localized with AGE in the striatum region of the PD mouse brain.

2-5. Protection of sRAGE and sRAGE-Secreting UCB-MSC Against AGE-Albumin-Induced Neuronal Cell Death

To examine the protective effects of sRAGE and sRAGE-secreting UCB-MSC on neuronal cell death, MIT assay was performed (Reference Example 8). Since the CS region is composed mainly of neurons, hippocampal nerve cells (HT22) for neuroprotective assay were prepared from the following three groups: control (untreated), AGE-albumin (50nM) treated (AA), and AGE-albumin (50 nM)+sRAGE (50 nM) treated (AA+sRAGE). The MIT assay results thus obtained are depicted in FIG. 9. As shown in FIG. 9, AGE-albumin-treated HT22 cells (AA) remarkably reduced in viability due to the induction of cell death whereas cell viability (100.96%) upon co-treatment with AGE-albumin and sRAGE (AA+sRAGE) was equivalent to or higher than the control (100%). These results indicate that sRAGE proteins protect neuronal cells from AGE-albumin-induced damage.

### Example 3

### Mechanism of Protection Against Neuronal Cell Death

3-1. MAPK Pathway Assay—p38, Erk1/2, and JNK Proteins Contribute to Cell Death in MAPK Pathway

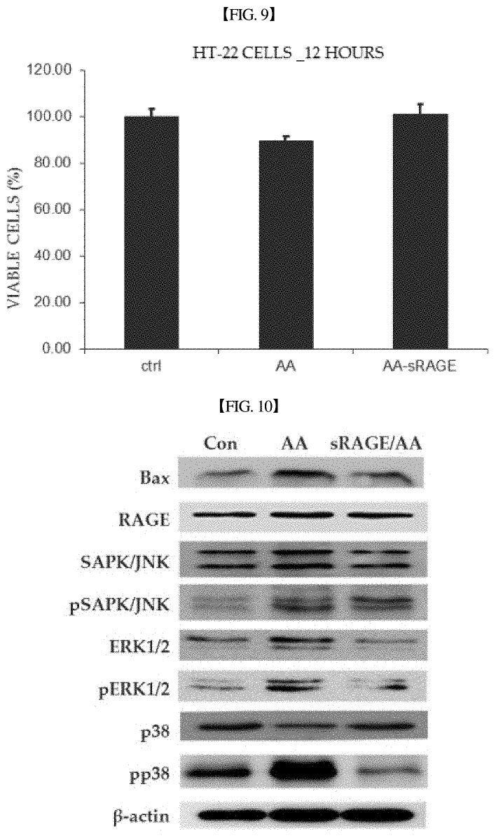

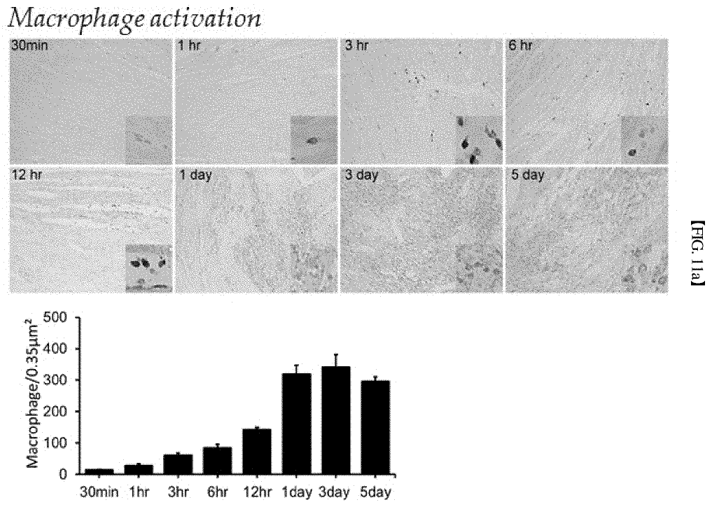

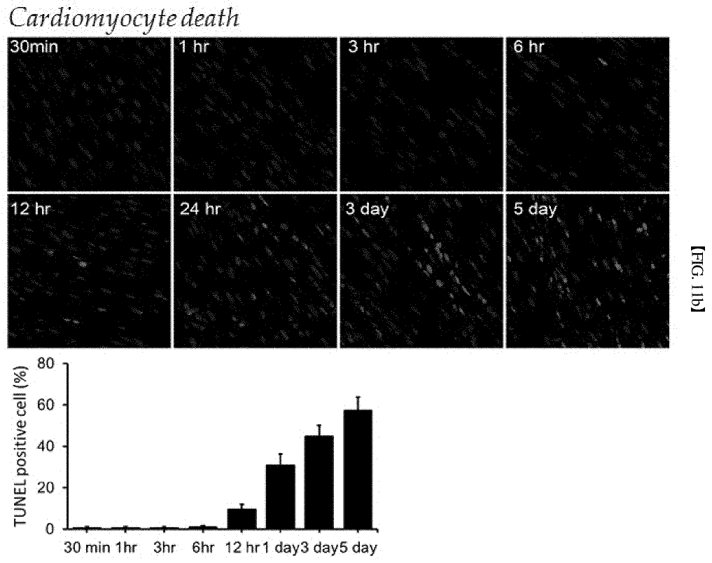

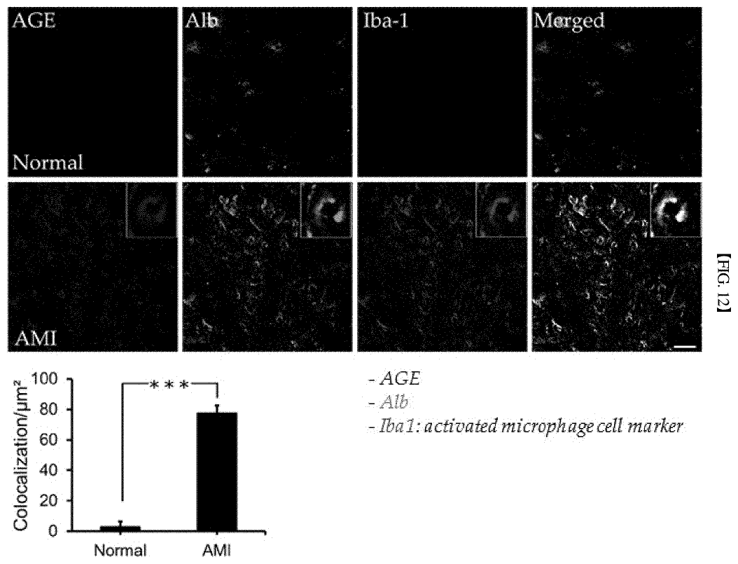

Changes of the main signaling pathway in the PD animal model were investigated at protein expression levels. To this end, tissues isolated from the CS region of PD mice were treated with AGE-albumin (50 nM) (AA) or AGE-albumin (50 nM)+sRAGE (50 nM), followed by western blotting analysis for the expression of proteins involved in the MAPK pathway (Reference Example 7). The result thus obtained is shown in FIG. 10. As shown in FIG. 10, JNK, p38, ERK1/2, and phosphorylated forms thereof were detected and expression levels of p38, Erk, and JNK were found to change. These results indicate that the three proteins (p38, Erk, and JNK) serve as contributors to neuronal cell death in PD mice, inducing neurodegeneration.

3-2. Bax Assay

Western blotting was performed to investigate the effect of sRAGE on the AGE-RAGE-dependent pathway (Reference Example 7). The results are depicted in FIG. 10. As shown in FIG. 10, Bax (apoptotic cell marker protein) was observed, and the expression of Bax was increased after AGE-albumin treatment, but decreased after co-treatment with sRAGE.

[Effect on Cardiovascular Disease]

### Example 4

### Synthesis and Release of AGE-Albumin in Macrophages of Heart Disease Patient

To examine whether phagocytes, capable of inducing myocardial infarction, synthesize and release AGE-albumin, expression levels of AGE-albumin in macrophages of myocardial infarction or limb ischemia model were measured using ELISA.

4-1. Cell Culture

For the in vitro study, immortal human macrophage cells (RAW264.7, Sigma-Aldrich) were employed. The macrophages were grown in DMEM (Dulbecco's modified Eagle's medium, Gibco)-high glucose supplemented with 10% heat-inactivated PBS (fetal bovine serum, Gibco) and 20 mg/ml gentamicin (Sigma-Aldrich) at 37° C. in a 5% CO₂ atmosphere and then cultured in a hypoxic condition.

4-2. Intracellular and Extracellular Levels of AGE-Albumin (ELISA)

Already synthesized albumin was removed from the medium using an anti-albumin antibody. Intracellular and extracellular (released to the culture medium) levels of AGE-albumin were measured using ELISA. Briefly, after hypoxic treatment of human macrophages, measurement was made using a cell lysate (0.5 μg protein) and a culture medium (0.1 mg protein). AGE-albumin was quantitatively analyzed using a rabbit anti-AGE antibody (1:1000, Abcam) and a mouse anti-human albumin antibody (1:800, Abcam). HRP-conjugated anti-mouse secondary antibody (1:1000, Vector Laboratories) was added to each well. In each well, color was developed with TMB (3,3′,5,5′-tetramethylbenzidine). The color development was stopped with an equal volume of 2M H₂SO₄. Absorbance at 450 nm was read on an ELISA plate reader (VERSA Max, Molecular Devices).

### Example 5

### Increased Synthesis and Release of AGE-Albumin in Human Macrophages by Myocardial Infarction

Myocardial infarction is known to accumulate over a long period of time by oxidative stress. Thus, examination was made to see whether the synthesis and release of AGE-albumin is induced by oxidative stress. To this end, human macrophages were treated with 0-1000 μM hydrogen peroxide (H₂O₂), which is an inducer of oxidative stress, followed by immunoblotting analysis using cell lysates. In addition, ELISA was performed to examine whether antioxidant treatment downregulates the expression of AGE-albumin in human macrophages.

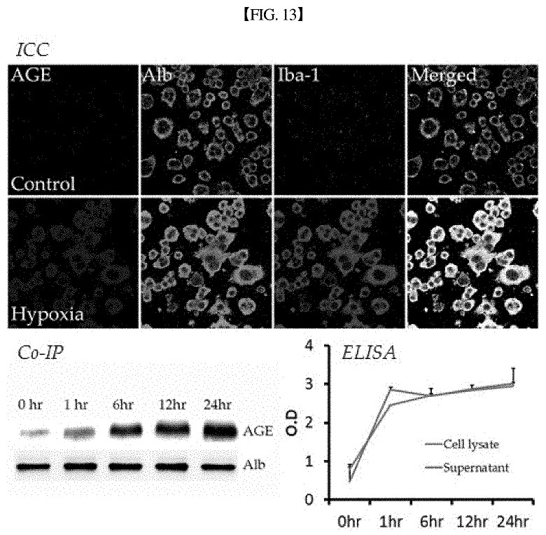

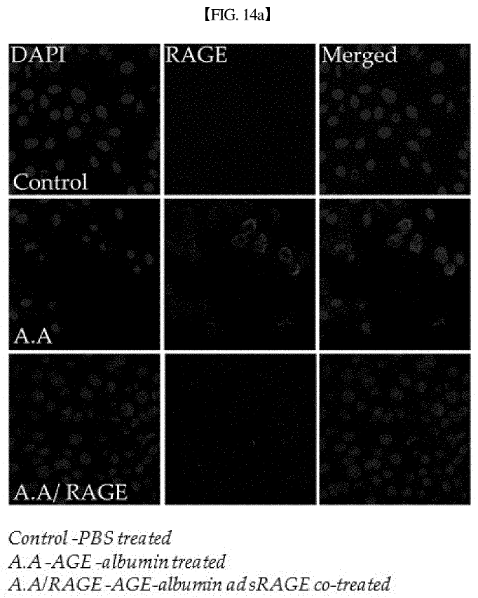

Results are depicted in FIG. 13.

As is apparent from data of FIG. 13, oxidative stress increased the synthesis and release of AGE-albumin in human macrophages.

### Example 6

### Distribution and Expression Position of AGE-Albumin in Rat Myocardial Infarction or Lower Limb Ischemia Model

6-1. Animal Model

Sprague-Dawley rats, each weighing 250-300 g, were prepared, and anaesthetized with a combination of ketamine (50 mg/kg) and xylazine (4 mg/kg). A 16-gauge catheter was inserted into the bronchus and connected with an artificial respirator. After the animal was fixed with a tape against a flat plate to secure the limbs and the tail, a 1-1.5 cm vertical incision was made left from the sternum, and the pectoralis major muscle was separated from the pectoralis minor muscle to ascertain the space between the 5^{th} and 6^{th} ribs. Then, the muscle therebetween was carefully 1 cm incised in a widthwise direction. A retractor was pushed in between the 5^{th} and 6^{th} ribs which were then separated further from each other. Since the upper part of the heart is typically covered with the thymus in rats, the thymus was pulled to the head using an angle hook to clearly view the heart. The figure of the left coronary artery was scrutinized to determine the range of artery branches to be tied. The LAD (left anterior descending artery) located 2-3 mm below the junction of the pulmonary conus and the left atrial appendage was ligated with 6-0 silk. Subsequently, the 5^{th} and 6^{th} ribs were positioned to their original places, and the incised muscle was sutured with MAXON 4-0 filament, followed by withdrawing air from the thoracic cavity through a 23-gauage needle syringe to spread the lungs fully. The skin was sutured with MAXON 4-0 filament. The catheter was withdrawn, and viscous materials were removed from the pharynx. After operation, a pain-relieving agent (Buprenorphine 0.025 mg/kg) was subcutaneously injected every 12 hours.

6-2. Immunohistochemistry (IHC)

Immunohistochemistry was conducted on heart tissues from normal or acute myocardial infarction (AMI) rats [S. M. Ahn et al., PLoS ONE 3, e2829 (2008)]. Normal or myocardial infarction heart tissues were fixed in 4% paraformaldehyde in a 0.1 M neutral phosphate buffer, cryopreserved overnight in a 30% sucrose solution, and then sectioned on a cryostat (Leica CM 1900) at a 10 μm thickness. Paraffin-embedded tissues were cut into 10 μm-thick sections, deparaffinized with xylene, and rehydrated with a series of graded ethanol. Normal goat serum (10%) was used to block non-specific protein binding. The tissue sections were incubated overnight at 4° C. with the following primary antibodies: rabbit anti-AGE antibody (Abcam), mouse anti-human albumin antibody (1:200, R&D System), and goat anti-Iba1 antibody (1:500, Abcam). Then, the tissue sections were washed three times with PBS before incubation for 1 hour at room temperature with one of the secondary antibodies: Alexa Fluor 633 anti-mouse IgG (1:500, Invitrogen), Alexa Fluor 488 anti-rabbit IgG (1:500, Invitrogen), and Alexa Fluor 555 anti-goat IgG (1:500, Invitrogen). After washing the secondary antibodies three times with PBS, coverslips were mounted onto glass slides using the Vectashield mounting medium (Vector Laboratories), and observed under a laser confocal fluorescence microscope (LSM-710, Carl Zeiss).

The results are depicted in FIG. 12.

As shown in FIG. 4, albumin (green) was co-localized with AGE (red) in rat heart cells after and before myocardial infarction. In addition, the blood monocytes from myocardial infarction rats were observed to have a wider distribution of albumin and AGE and a higher expression level of AGE-albumin, compared to those from normal rats.

### Example 7

### Suppressive Effect of Soluble RAGE (sRAGE) on AGE-Albumin Synthesis in Myocardial Infarction Model (In Vivo)

To confirm the increase of RAGE in myocardial infarction model and the suppressive effect of sRAGE on RAGE, immunohistochemistry was conducted for RAGE (red) and DAPI (blue) and the distribution and expression position of AGE-albumin was observed under a laser confocal fluorescence microscope.

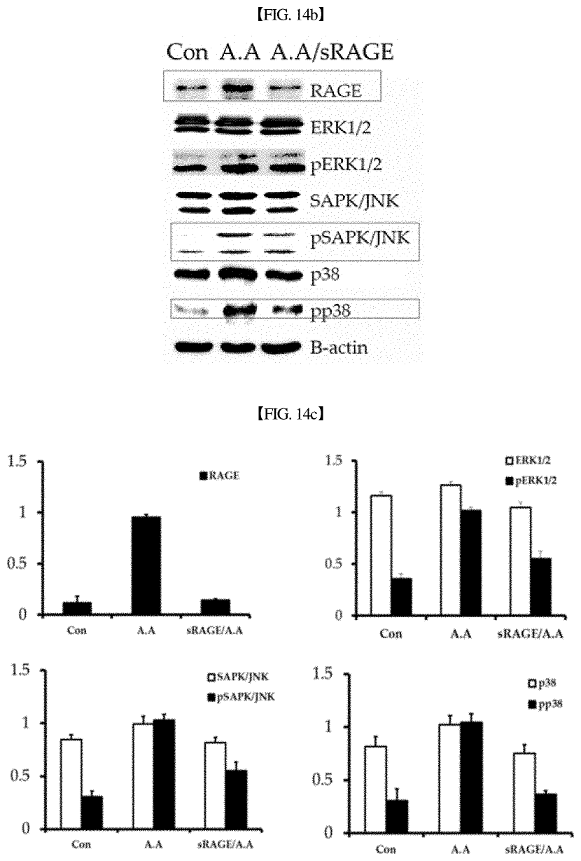

The result is depicted in FIG. 14.

As viewed in FIG. 14, an increase or decrease in RAGE level was detected in the cells in rat blood monocytes after or before administration of sRAGE to myocardial infarction rats. This change was found to be most greatly influenced by pSAPK/JNK and pp38 in the MAPK pathway.

### Example 8

### Induction of Cell Death by AGE-Albumin in Cardiomyocyte

Stress-activated MAPK (Mitogen-Activated Protein Kinase) is reported to play a critical role in neuronal cell death. Hence, experiments were carried out to examine whether AGE-albumin directly induce cell death in human cardiomyocytes, as follows.

8.1. Human Cardiomyocyte Culturing

Cardiomyocytes were suspended in DMEM (culture medium) containing 5% FBS, 5% HS (horse serum), 20 μg/ml gentamicin and 2.5 μg/ml amphotericin B, plated at a density of 1×10⁶cells/ml (10 ml) into 10-cm culture dishes, and maintained at 37° C. in a 5% CO₂/95% atmosphere in an incubator. After 2-3 weeks of in vitro culture, the cells were treated with AGE-albumin and used in analyzing apoptosis-related properties.

8.2. Cell Viability (MTT Assay)

Human cardiomyocytes were seeded at a density of 2×10³ cells/well into 96-well plates. When reaching 80% confluence, the human cardiomyocytes were treated with various concentrations (0, 0.01, 0.1, 1, 10, 20 μg/ml) of albumin or various concentrations (0, 0.5, 1, 5, 10 mg/ml) of AGE-albumin After 24 hours of treatment, the cells were rinsed with PBS and examined for viability using an MIT [3-(4,5-dimethylthiazol-2-yl)-2,5-diphenyl tetrazolium bromide] assay. Absorbance in each well was read at 540 nm on a 96-well plate reader (VERSA Max, Molecular Devices).

The results are depicted in FIG. 14.

As shown in FIG. 14, when human cardiomyocytes were treated with AGE-albumin, cell viability decreases with an increase in AGE-albumin concentration, indicating that AGE-albumin induces cell death. In contrast, when primary human cardiomyocytes were treated with albumin alone, the cell viability remained almost unchanged irrespective of albumin concentration, indicating that albumin does not induce cell death.

To investigate the protective effect of soluble RAGE on cardiomyocyte apoptosis, cardiomyocytes were treated with sRAGE alone, AGE-albumin alone, or sRAGE/AGE-albumin and measured for viability.

The results are depicted in FIG. 14.

As shown in FIG. 14, human cardiomyocytes increased in cell viability and were not prone to apoptosis when co-treated with sRAGE and AGE-albumin, indicating that sRAGE has a protective effect on cardiomyocyte apoptosis.

### Example 9

### Technology for Generating Growth Factor-Secreting Stem Cell Applicable to Human

Generation Technology for sRAGE-Secreting Cell by Using CRISPR/Cas9 RNP

Generation of sRAGE-Secreting Cells

To begin with, the RAGE gene (GenBank Accession No. NM_001206940.1) was inserted into a pZDonor vector (Sigma Aldrich) to construct a recombinant pZDonor vector carrying the sRAGE gene (see FIG. 15a). In addition, AAVS1-targeting CRISPR/Cas9 RNP (ToolGen Inc.) was prepared (Cas9: Streptococcus pyogenes-derived Cas9 protein; sgRNA sequence for AAVS1: gucaccaauccugucccuag; refer to General Formula 3 supra, with respect to the entire sequence).

The above-prepared vector carrying AAVS1-targeting CRISPR/Cas9 RNP and the recombinant pZDonor vector carrying sRAGE were co-transfected into human umbilical cord mesenchymal stem cells (Medipost).

CRISPR/Cas9 RNP cleaves an AAVS site on cell genomic genes to insert a desired gene (e.g., sRAGE gene) into the cleaved site, thereby generating sRAGE-secreting cells. The sRAGE secretion of the generated cells was examined by western blotting, ELISA, and fluorescent immunostaining (Flag), and the results are depicted in FIGS. 5b and 5c.

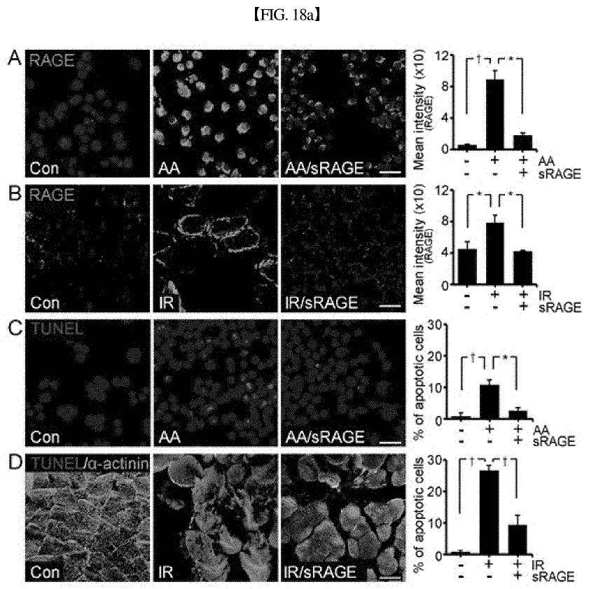

In addition, gene editing (Indel: insertion and/or deletion) efficiency of the above prepared CRISPR/Cas9 RNP was tested in Jurkat cells and the results are depicted in FIG. 16 (none: mock transfection; sgRNA #1: transfected with number 1 sequence-targeting guide RNA alone; sgRNA #2: transfected with number 2 sequence-targeting guide RNA alone; Sp.cas9 only: transfected with cas9 protein alone; aRGEN1: transfected with number 1 sequence-targeting gRNA plus cas9; aRGEN2: transfected with number 2 sequence-targeting gRNA plus cas9; dRGEN1: transfected with number 1 sequence-targeting gRNA plus cas9-carrying plasmid; dRGEN2: transfected with number 2 sequence-targeting gRNA plus cas9-carrying plasmid)

The results depicted in FIGS. 15 and 16 were obtained using the following assays:

Standardization Assay of Stem Cell and Specific Material-Secreting Cell

RT-PCR Assay

After RNA isolation using Trizol, cDNA was synthesized using an olig-dT primer and a reverse transcriptase. cDNA synthesis started with reverse transcription at 42° C. for one hour, followed by thermal treatment at 95° C. for 10 min to stop the enzymatic activity. Primers for a gene of interest were designed and used for PCR (primers: Fwd: 5′-cggaactctgccctctaacg-3′; Rev: 5′-tgaggaagagacttgcagct-3′).

Western Blot

The protein concentration in an isolated protein solution was measured by BCA assay and a predetermined amount of the protein solution was run on a 10% SDS-PAGE gel by electrophoresis before transfer onto a PVDF membrane. This membrane was incubated with a primary antibody (Sigma Aldrich) at 4° C. for 12 hours and then washed to remove the unbound antibody. Subsequently, incubation with an HRP-conjugated secondary antibody (Vector Laboratories) was done at room temperature for one hour. After completion of the reaction, protein expression was analyzed with ECL (Amersham).

Immunocytochemistry-Fluorescent Staining

Fixed cells were reacted with a primary antibody at 4° C. for 12 hours and washed, followed by incubation with fluorescein-conjugated goat anti-rabbit IgG at room temperature for one hour. The cells thus stained were mounted on a glass slide and observed under a Zeiss confocal microscope.

Characterization of Generated Human Umbilical Cord-Derived, Growth Factor-Secreting Stem Cells

After being cultured, the generated, vascular endothelial growth factor-secreting functional stem cells were evaluated for proliferative activity, cell-labeling marker (immunophenotype) and multipotency, and migration and secretory function, using stem cell characterization assays. Selection was made of highly effective sRAGE-secreting stem cells according to predetermined criteria The selected sRAGE-secreting stem cells were called sRAGE-UC-MSC.

### Example 10

### Protective Effect of sRAGE-UC-MSC on Cardiomyocyte Death in Myocardial Infarction Model

In Vivo Assay

To investigate the protective effect of sRAGE on cardiomyocyte death in a myocardial infarction model, a rat myocardial infarction model was constructed and the sRAGE-UC-MSCs selected in Example 6 were injected to the tissues of the model (injection dose: 10 μl*3 times, a total of 30 μl, 1×10⁶ cells in 30 μl). The cardiomyocytes were stained with cresyl violet and counted under a microscope.

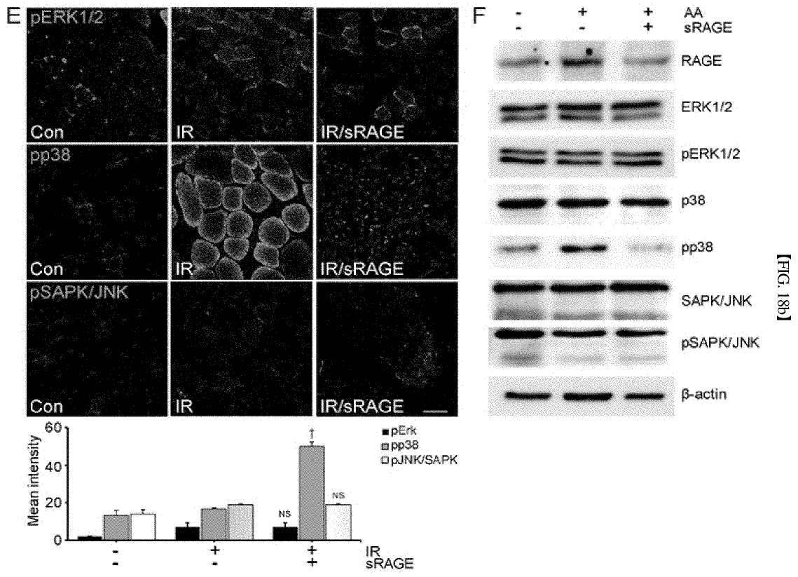

The result is depicted in FIG. 17.

As shown in FIG. 17, the treatment of rat heart tissues with sRAGE-UC-MSCs reduced the myocardial infarction area and the fibrosis range.

### Example 11

### Protective Effect of sRAGE-UC-MSC on Myocyte Death in Lower Limb Ischemia Model

In Vivo Assay: Construction of Animal Model (Rat Lower Limb Ischemia Model)

As experimental animals, male Balb/c-nu mice were used. Animal model construction was conducted in a clean and sterile environment under the anesthesia by N₂O:O₂=1:1 (v:v), isoflurane inhalation.

After anesthesia, incision was made about 2 cm on the skin. Then, 3-0 surgical silk was applied to an accurate site (5-6 mm below iliac arteries or superficial femoral arteries and inguinal ligament) for ligation, followed by closing the skin with a skin clip.

To examine the protective effect of sRAGE on lower limb myocyte death in lower limb ischemia model, a rat lower limb ischemia model was constructed and sRAGE (protein) was injected into the tissue (injection dose: a total of 8 μl containing 0 8 μg of sRAGE protein). The myocytes was stained against RAGE, TUNEL, and a-actinin and observed under a confocal microscope.

The results are depicted in FIGS. 18a and 18b. In FIG. 18a (A, C: in vitro; B, D: in vivo) and 18b, AA stands for Age-albumin administered group, IR for ischemia reperfusion model group, and sRAGE for sRAGE (protein) administered group.

As shown in FIGS. 18a and 18b, the treatment of the rat lower limb with sRAGE-UC-MSC reduced the expression of RAGE and TUNEL, and pp38 was found to be involved in the reduced expression.

### Example 12

### Preparation and Characterization of sRAGE-iPSC

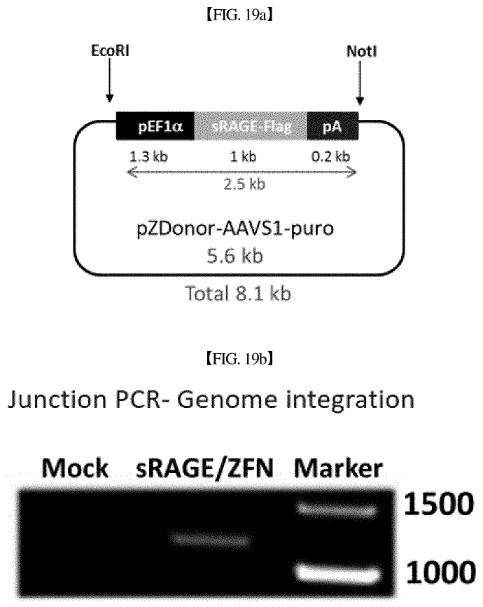

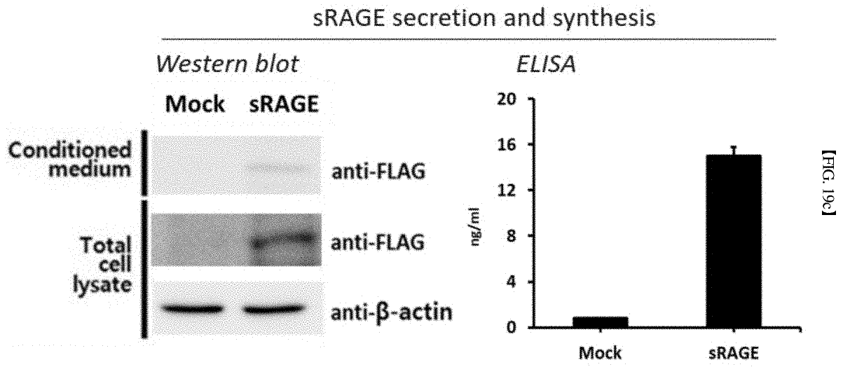

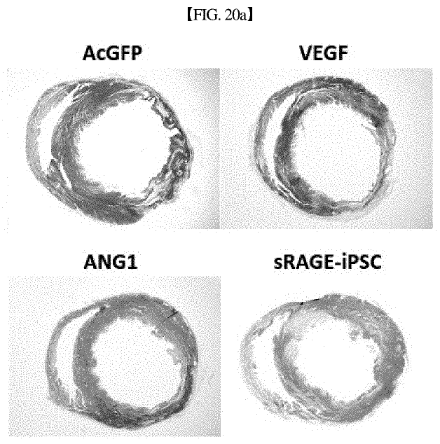

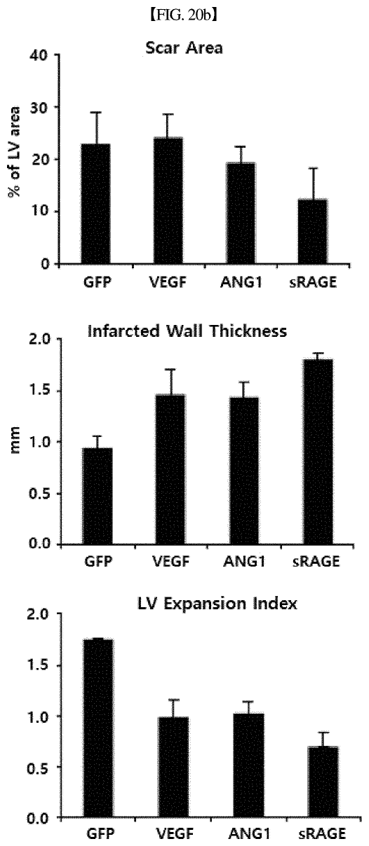

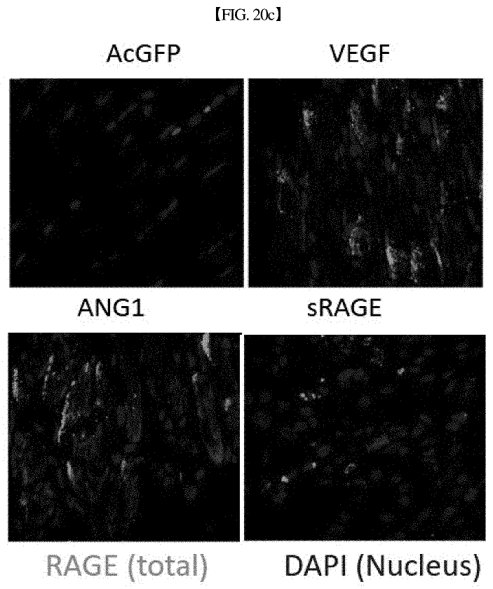

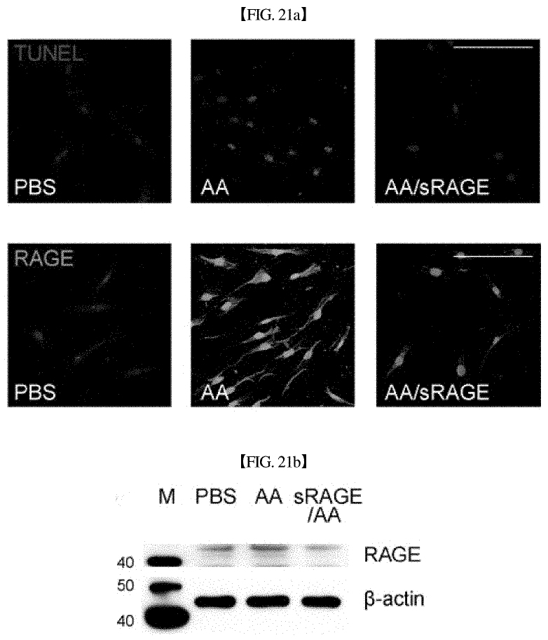

In order to generate sRAGE-secreting iPSCs, sRAGE donor vector constructed by cloning a human EF1-α promoter, an sRAGE-encoding sequence, and poly A tail into the pZDonor vector (Sigma-Aldrich) (see FIGS. 1A and 19a) was transfected, together with the CRISPR/CAS9 RNP, into iPSCs. The guide RNA was designed to target a safe harbor site known as AAVS1 on chromosome 19 (Cas9: derived from Streptococcus pyogenes (SEQ ID NO: 4), target site of sgRNA: gtcaccaatcctgtccctag (SEQ ID NO: 7)). Transfection was performed using a 4D nucleofector system (Lonza). Transfection conditions were as set forth in the Lonza protocol (cell type ‘hES/H9’) on the website. Electroporation was performed using P3 primary cell 4D nucleofector X kit L (Lonza, V4XP-3024). sRAGE-secreting iPSCs were created by transfecting 15 μg of Cas9 protein, 20 μg of gRNA, and 1 μg of the sRAGE donor vector into 2×10⁵ human iPSCs (Korean National Stem Cell Bank).

Three days after transfection, genomic DNA was isolated from the transfected iPSCs to determine the knock-in (KI) of sRAGE in the genomic DNA of the iPSCs. PCR primers were prepared with MVS1 Fwd (iPSC itself sequence) and Puro rev (insertion sequence) (AAVS1 FWD primer: CGG AAC TCT GCC CTC TAA CG; Puro Rev primer: TGA GGA AGA GTT CTT GCA GCT).

PCR is performed at 56° C. with 30 cycles. Following electrophoresis, the bands were observed under UV light. The obtained result is shown in FIG. 19b. FIG. 19b shows that the gene of sRAGE was successfully integrated at the MVS1 site.

Expression and secretion levels of sRAGE were confirmed by immunoblotting and ELISA.

First, immunoblotting was performed as follows: a whole cell lysate was prepared in RIPA (radio immunoprecipitation assay) lysis buffer (ATTA, WSE7420) and protease inhibitor cocktail (ATTA, WSE7420), followed by sonication. The prepared cell lysate was centrifuged at 17,000×g for 20 minutes at 4° C., and the supernatant was collected. Equal amounts (30 30 μg) of proteins were separated on 10% polyacrylamide gel and the separated proteins were transferred onto a nitrocellulose membrane (Millipore) at 200 mA for 2 hours. The membrane was treated with 5% non-fat skim milk for 1 hour at room temperature to block nonspecific antibody binding. The membrane was incubated overnight at 4° C. with a primary protein-specific antibody (Sigma, F-7425) and b-actin (Abeam, ab8227) and then with a secondary antibody at room temperature for 1 hour. After several washes, proteins were detected using enhanced chemiluminescence (ECL).

ELISA was performed as follows: the total soluble RAGE secreted was quantified using a human soluble receptor advanced glycation end products (sRAGE) ELISA kit (Aviscera Bioscience, SK00112-02). Samples and 100 μl of the standard solution were added (in the reverse order of serial dilutions) to 96-well microplates pre-coated with a human sRAGE antibody and containing 100 μl of a diluted complete solution per well. The plates were then covered with a seal and incubated for 2 hours on a micro plate shaker at room temperature. After incubation, the solution was completely aspirated and washed four times with a wash solution. A dilution of a detection antibody in a working solution was added in an amount of 100 μl to each well after which the plate was covered with a seal and incubated on a microplate shaker at room temperature. Aspiration and washing was repeated again. A horseradish peroxidase (HRP)-conjugated secondary antibody was added in an amount of 100 μl to each well, followed by incubation for 1 hour on a microplate shaker at room temperature under a light-tight condition. Aspiration and washing was repeated again. Finally, 100 μl of the substrate solution is added to each well and reacted for 5-8 minutes before 100 μl of the stop solution was added to terminate the reaction. Optical density was measured using a micro plate reader set at 450 nm.

The results obtained by performing western blot and ELISA are shown in FIG. 19c. As can be seen from the western blot results of FIG. 19c, the expression of Flag was observed in pzDonor vector-transfected sRAGE-iPSCs. As understood from the ELISA result of FIG. 9b, which accounts for the total expression level of sRAGE secreted to the medium, sRAGE was detected at a concentration of 15.6 ng/ml in the culture medium of sRAGE-iPSCs, which was markedly high, compared to the level of sRAGE detected in the medium of mock-iPSC, 0.8 ng/ml.

### Example 13

### Myocardial Infarction (MI) Modeling and sRAGE-iPSC Transplantation

Myocardial infarction was introduced into Sprague-Dawley male rats (age of 8 weeks), each weighing 290-330 g, by conducting MI and reperfusion. Briefly, the rats were intubated and ventilated with a volume-cycled small-animal ventilator. Anesthesia was maintained with 5% isoflurane during the operation. The heart was exposed for 40 minutes through a left lateral thoracotomy, and the left anterior descending coronary artery (LAD) was ligated by 6-0 polypropylene thread. After reperfusion, 10 μl of PBS was injected, alone or together with GFP-iPSC or sRAGE-iPSC cells (1×10⁶ cells), into the peri-infarction zone or an infarction area through a Hamilton syringe. The muscle layer and the skin were sealed and left for recovery. A sham-operated group underwent the same experimental procedure, but for ligation and cell transplantation. In order to prevent transplant rejection, the cell-transplanted rats were administered cyclosporine A (10 mg/kg/day). All the animal experiments were performed under the approval of the Institute Animal Care and Use Committee of the Lee Gil Ya Cancer and Diabetes Institute at Gachon University (#LCDI-2014-0020).

Four weeks after cell transplantation, the animals were sacrificed. The heart was excised and perfused with PBS and iced 4% paraformaldehyde through the right carotid artery. The tissue was fixed overnight at 4° C. in 4% paraformaldehyde (PFA, Sigma-Aldrich, 158127) before a dehydration procedure. After dehydration, the tissue was cleared twice with xylene for 1.5 hours in each time and embedded at 60° C. in paraffin. The paraffin-embedded heart tissue was cut into sections 7 μm thick.

H&E and Masson trichrome staining was performed to measure infarct sizes, anterior wall thicknesses, and fibrosis ratios. H&E and Masson's trichrome-stained sections were observed under an optical microscope and the collagen-delegated infarct ratio was calculated and analyzed by the blinded investigator. The sections for measuring infarcted area sizes and other parameters were prepared by slicing the tissues in the short axis direction at an intermediate position between the cardiac apex and the ligation site. The infarction size was evaluated, on the basis of the results using the following formulas:

% infarct size=(infarct areas/total left ventricle (LV area))×100%

infarct thickness=(anterior wall (infarct wall thickness)/septal wall thickness)×100

Viable LV area=total LV myocardial area−infarct myocardial area

The results are depicted in FIGS. 20a to 20c, showing that treatment with sRAGE-secreting iPSC inhibited cardiomyocyte death in the ischemic reperfusion injured rat heart. In greater detail, FIG. 20a show sizes of myocardial infarction areas 28 days after the operation and GFP-iPSC or sRAGE-iPSC transplantation as analyzed by Masson's trichrome staining. In FIG. 20a, the infarction damage-induced fibrosis areas appear blue while cardiomyocytes are indicated by red colors. The results of FIG. 20a were quantitated using Image J software. On the basis of the quantitation, percentages of fibrosis areas and infarcted wall thicknesses in the LV cross sectional area were calculated and are depicted in FIG. 20b. A significant reduction of fibrosis area was observed in the sRAGE-iPSC-administered group, compared to the iPSC-, VEGF-iPSC- or ANG1-iPSC-administered group. In addition, as shown in FIG. 20c, the level of RAGE was significantly reduced in the sRAGE-iPSC-treated group, compared to the VEGF- or ANG1-treated group.

### Example 14

### Protective Effect of sRAGE-Secreting iPSC on Stem Cell

Immunohistochemistry assay revealed that the level of TUNEL was increased in the AGE-albumin (AA)-treated iPSC, but decreased when the iPSCs were co-cultured with sRAGE-secreting iPSCs (sRAGE-iPSC) (see FIG. 21a). In addition, western blots against RAGE in PBS-, AA-, and sRAGE-iPSC-treated groups are given in FIG. 21b, showing that following AA treatment, RAGE was expressed at a decreased level in the iPSCs co-cultured with sRAGE-iPSC. The data indicate that sRAGE-secreting iPSC has a protective effect on other stem cells including iPSCs (particularly, stem cell protection effect in the AGE-albumin accumulation environment, such as myocardial infarction) and thus can improve a stem cell therapy product when used in combination therewith, suggesting a use of sRAGE-secreting iPSC in combination therapy with other stem cell therapy products.

1-9. (canceled)

10. A method of suppressing the secretion of AGE (advanced glycation end-products)-albumin or inhibiting AGE-albumin-induced cell death (apoptosis), the method comprising a step of administering a soluble receptor for advanced glycation end products (sRAGE)-secreting stem cell or a cell culture thereof to a subject in need of repressing the secretion of AGE-albumin or inhibiting AGE-albumin-induced cell death.

11. The method of claim 10, wherein the stem cell is at least one selected from the group consisting of embryonic stem cells, adult stem cells, and induced pluripotent stem cells (iPS cells).

12. The method of claim 11, wherein the stem cell is an induced pluripotent stem cell or a mesenchymal stem cell.

13. A method of preventing or treating a neurologic disease or a cardiovascular disease, the method comprising a step of administering a soluble receptor for advanced glycation end products (sRAGE)-secreting stem cell or a cell culture thereof to a subject in need of preventing or treating a neurologic disease or a cardiovascular disease.

14. The method of claim 13, wherein the stem cell is at least one selected from the group consisting of embryonic stem cells, adult stem cells, and induced pluripotent stem cells (iPS cells).

15. The method of claim 14, wherein the stem cell is an induced pluripotent stem cell or a mesenchymal stem cell.

16. The method of claim 13, wherein the neurologic disease is Parkinson's disease (PD), amyotrophic lateral sclerosis (ALS, Lou Gehrig's disease), frontotemporal dementia (FTD), dementia with Lewy bodies (DLB), corticobasal degeneration, multiple system atrophy (MSA), progressive supranuclear palsy (PSP), Huntington's disease (HD), or spinal cord injury

17. The method of claim 13, wherein the cardiovascular disease is stroke, myocardial infarction, angina pectoris, limb ischemia, hypertension, or arrhythmia.

18. A method of protecting an isolated stem cell, the method comprising a step of co-culturing the isolated stem cell with an isolated stem cell secreting soluble receptor for advanced glycation end products (sRAGE).
